(19) United States
(12) Patent Application Publication
(10) Pub. No.: US 2026/0166211 A1
(43) Pub. Date: Jun. 18, 2026

(54) SYSTEM FOR REMOVING PROTEIN BOUND UREMIC TOXINS DURING HEMODIALYSIS

(71) Applicant: University of Washington, Seattle, WA (US)

(72) Inventors: Bruce J. Hinds, III, Seattle, WA (US); Yushi Zang, Seattle, WA (US)

(73) Assignee: University of Washington, Seattle, WA (US)

(21) Appl. No.: 19/126,462

(22) PCT Filed: Oct. 31, 2023
(86) PCT No.: PCT/US2023/078282

§ 371 Date: May 1, 2025

Related U.S. Application Data
(60) Provisional application No. 63/382,006, filed on Nov. 2, 2022.

(51) Int. Cl. A61M 1/34

(52) U.S. Cl. CPC A61M 1/3482; A61M 1/3486; A61M 2202/0021; A61M 2202/0042; A61M 2202/0445

(57) ABSTRACT

Systems and methods for physiologically safe removal of protein-bound uremic toxins (PBUTs) from blood proteins, including albumin, during dialysis. A dialysis system for removal of PBUTs includes a protein-bind column operably connected to at least a portion of a plasma stream. During use, the column captures albumin and PBUTs complexed with the captured albumin are released from the albumin with a toxin release step into a waste stream or dialyzer. The captured albumin is then released from the column with a protein release step and the released albumin reenters the plasma stream and the patient's body. Compositions used for release steps, including the protein release step, are physiologically safe for entry into the patient's body. Since the toxins are removed from the albumin, the patient has a lower risk of health complications due to the presence of PBUTs in the bloodstream.

### CROSS-REFERENCE TO RELATED APPLICATION

This PCT application claims the benefit of U.S. provisional patent application number 63/382,006 filed on Nov. 2, 2022, the contents of which is incorporated herein by reference in its entirety for all purposes.

### BACKGROUND

In-center hemodialysis is lifesaving treatment for 85% of patients with end stage renal disease (ESRD), which carries a significant global health burden and affects about 300,000 Americans each year. Hemodialysis carries an annual cost of 28 billion USD in the United States or about 90,000 USD per patient. However, even with this large expense, the five-year survival rate is only 35-42% based on data from 2005-2015, with cardiac failure and infections being the leading causes of mortality, and there is a risk of health complications due to the presence of protein-bound uremic toxins (PBUTs) in dialyzed blood.

Conventional kidney hemodialysis is effective for small molecule toxin removal using ˜0.5 kDa molecular weight cutoff (MWCO) membranes. However. PBUTs are difficult to remove with conventional dialysis since the protein-toxin complex is too large (˜66.5 kDa) to pass through dialysis membranes. In addition, even if the protein-toxin complex were removed from the blood, the patient would lose albumin in the process which would cause further complications if the albumin were not replaced in the body, a further expense.

PBUTs have a low unbound fraction, which proportionately lowers their removal efficiency with respect to other small molecule solutes. For instance, indoxyl sulfate (IS), which is associated with cardiovascular disease, has 95% bound to albumin at equilibrium. Some treatments allow a fraction of human albumin (HA), with and without toxins, to be removed in dialysis using high MWCO (˜70 kDa) membrane, but the approach suffers from potential malnutrition and hypoalbuminemia. Despite the addition of activated carbon to hemodialyzer cartridges as part of efforts to remove PBUTs, PBUT levels remain far above non-uremic levels with these treatments.

HA is a heart-shaped protein made of three homologous helical domains (I-III) that can be further divided into subdomains A and B (FIG. 1). Fatty acids bind to multiple binding sites spread over all three domains. PBUT molecules, such as IS and p-cresyl sulfate, are associated with cardiorenal syndrome and are made of an ionic portion and an aromatic portion that make it strongly bind to hydrophobic cavities with cationic entrances in subdomain II-A and III-A. The Sudlow site IL (S-II), where indoxyl sulfate mainly binds to, is located at subdomain III-A and also contains two fatty acid binding sites. Thus, IS and fatty acids may compete in binding to the S-II site.

While healthy kidneys have specialized molecular pumps to selectively remove anionic-aromatic compounds from the S-II site, conventional kidney dialysis lacks this ability. Strategies such as the Molecular Adsorbents Recycling System (MARS) employs an albumin-containing dialysate system to promote removal of PBUTs. The externally supplied HA is in a dialysate loop and carries PBUTs from the blood loop to an activated carbon (AC) “sink” for removal. However, such processes are costly due to the requirement for added HA, and lack any improvement in toxin selectivity. An absorbent, AST 120, was developed to target food sourced toxin precursors that exist in the digestive system. While AST lowers the patient's serum IS level by 40% after 12 weeks of treatment, an undesirable amount of absorbent (30 capsules daily) is needed to be administrated orally. Metal organic framework (MOF) was proposed as a better toxin-selective absorbent, and while some studies showed high removal rates of plasma toxin with high selectivity, the majority of toxin was still bound to proteins and the toxin removal efficiency was not improved compared to conventional dialysis. Other approaches directly focus on HA cleansing by using cyclic electromagnetic field or adding PBUT binding competitors to conventional dialysis systems and showed some improved efficiency, but have technical challenges or limitations. For conventional protein separation processes, HA is typically purified from blood plasma by use of chromatography columns functionalized with aromatic and charged Cibacron blue dye that binds to S-II sites and released by highly concentrated thiocyanate. With previous systems, extensive ion exchange rinsing to remove thiocyanate is needed for a physiologically safe product, which is impractical in a patient setting.

Therefore, there remains a significant need for efficient and physiologically safe PBUT removal during hemodialysis. The present disclosure addresses this and other long-felt and unmet needs in the field.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The disclosure provides material systems and methods for release of PBUTs from HA, then release of cleaned HA back into the blood stream with a physiologically safe release agent. The disclosed strategies reduce PBUTs during hemodialysis treatments by binding HA-toxin complexes to a surface, releasing toxin to a waste stream and/or dialyzer, then releasing the cleaned albumin back into the blood plasma stream. A key advantage of the disclosed approaches is the use of blood plasma compatible chemistry, particularly for the release step during which HA is released from the surface.

In an aspect, the disclosure provides a dialysis system, comprising an immobilized ligand configured to competitively bind a toxin binding site of a protein and displace a toxin from the toxin binding site of the protein into a first eluate. In a general sense, the dialysis system is configured for return of the protein, with reduced or no toxin bound, to blood of a subject.

In another aspect, the disclosure provides a dialysis system, comprising a protein-bind column operably connectable to a plasma fluid stream at an inlet thereof and at an outlet thereof. The protein-bind column comprises a resin that comprises an immobilized ligand configured to bind a toxin binding site of an albumin protein and displace a uremic toxin from the toxin binding site of the albumin protein into a first eluate. The system further comprises a plasma fraction membrane cartridge, positioned upstream of the protein-bind column, configured to divert a portion of plasma of whole blood of a subject into the plasma fluid stream. After the toxin is removed from albumin of the plasma fluid stream, the plasma fluid stream is returned to blood of a subject.

In another aspect, the disclosure provides a hemodialysis method for removal of PBUTs from whole blood. The method comprises separating a toxic plasma fraction from whole blood of a subject in need of dialysis, binding a toxin-protein complex of the toxic plasma fraction to an immobilized ligand, separating a toxin from the toxin-protein complex such that the toxin flows to a first eluate and a protein of the toxin-protein complex remains bound to the immobilized ligand, dialyzing the first eluate to remove the toxin and produce a clean first eluate, eluting the protein from the immobilized ligand with a biocompatible release agent such that the protein flows to a second eluate, removing at least a portion of the biocompatible release agent from the second eluate to form a clean second eluate, dialyzing and combining the clean first eluate and the clean second eluate to produce a clean plasma fraction, and returning at least a portion of the clean plasma fraction to blood of the subject. The steps can be performed in any suitable sequence, optionally with two or more steps being performed simultaneously. The method can be performed using any suitably configured dialysis system, and is not limited to the example systems of the disclosure.

In yet another aspect, the disclosure provides a composition comprising a clean plasma fraction, as well as a method for treating end stage renal disease (ESRD) in a subject in need thereof. The method of treatment comprises administering the composition that includes the clean plasma fraction to the subject. Since the composition is partially, mostly, or completely free of PBUTs, the subject has a significantly lower risk of a variety of health problems associated with the presence of PBUTs in the blood.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings.

FIG. 1 shows human albumin (HA) structure with binding sites labeled.

FIG. 2A shows a dialysis system, which includes a hemodialysis cartridge operably connected with a dialysis loop.

FIG. 2B shows an example toxin removal dialysis system of the disclosure, which includes a toxin removal loop in combination with a hemodialysis cartridge and dialysis loop.

FIG. 3A shows a first example toxin removal dialysis system of the disclosure, which includes combined dialysis of a stream that includes post-toxin removal plasma and whole blood and use of a small molecular weight (SMW) cutoff for recirculation of bloodborne small molecular weight compounds through the system.

FIG. 3B shows a second example toxin removal dialysis system of the disclosure, which includes separated dialysis of post-toxin removal plasma and whole blood.

FIG. 4A shows an example chemical grafting process for surface plasmon resonance (SPR) chips using carboimide coupling (Ligand) and click chemistry coupling (Ligand′).

FIG. 4B shows an example Cibacron blue chromatography bead.

FIG. 5A shows a schematic of an example adsorption column.

FIG. 5B shows a schematic of an example assembled plasma/linoleic acid (LA) filter.

FIG. 5C shows a schematic of an example Cibacron blue functionalized bead column.

FIG. 5D shows a schematic of an example sonicator with carrier flow and LA flow.

FIG. 5E shows a schematic of an example LA filter configuration for operation and inline cleansing.

FIG. 5F shows a schematic of an example circuit diagram for a toxin removal dialysis system of the disclosure.

FIG. 6A shows a representative SPR absorption curve of albumin on a functionalized (Direct Blue 71) SPR sensor chip, according to aspects of the disclosure.

FIG. 6B shows a Langmuir isotherm curve from the representative SPR absorption curve of FIG. 6A, according to aspects of the disclosure.

FIG. 7 shows a representative SPR signal response for HA adsorption and desorption (in cycles), according to aspects of the disclosure. Light gray curves represent control channels with continuous buffer rinse after initial HA adsorption. Dark gray curves represent the signal shift as HA is adsorbed/desorbed as different solutions are flowed through. The adsorption/desorption test was performed in repeating sequence with different solutions indicated by different colored arrows and notations (B: phosphate buffer; HA: human albumin; LA: linoleic acid; R: conventional release with NaSCN). Labeled numbers are SPR signal shift representing the amount of stably adsorbed albumin and the amount of released albumin after release agent rinse. Calculated release of bound albumin by release agent is shown in percentage.

FIG. 8A shows a representative SPR curve of HA-IS complex adsorption and desorption in cycles on a CB-functionalized SPR chip, according to aspects of the disclosure. Dark gray curve represents the absorption/desorption test performed with indicated release chemicals while light gray are buffer reference channels after HA-IS adsorption. The test was performed in repeating sequence with different solutions indicated by different colored arrows and notation (B: phosphate buffer; HA-IS: human albumin indoxyl sulfate complex: LA: linoleic acid: R: NaSCN). Labeled numbers are SPR signal shift representing the amount of stably adsorbed albumin and the amount of unreleased albumin after release agent rinse. Calculated releasability of bound albumin is shown in percentage.

FIG. 8B shows UV-vis absorbance 280 nm (dark curve) and BCA assay (light curve) for eluent solutions with respect to cumulative volume of eluent passing through 0.52 mL CB-agarose beads, according to aspects of the disclosure. Vertical black lines demarcate eluent regions while acronyms indicate eluent composition: ‘B’ for buffer. “LA” for linoleic acid, “HA-IS” for 200 μM human serum albumin with 200 μM indoxyl sulfate in buffer, and “NaSCN” for 2 μM NaSCN in buffer. Lines are guides to the eye.

FIG. 9A shows (upper left, lower left) HA-IS solution fluorescence signal profiles and (upper right, lower right) fluorescence signal peak locations at constant HA (50 μM) and varying IS concentrations (50, 40, 20, 10, 5, 2.5, 1, 0 μM), according to aspects of the disclosure. (upper left, upper right) HA-IS is dissolved in buffer and (lower left, lower right) HA-IS is dissolved in buffer containing LA micelles. Due to dilution needed for assay, 50 μM corresponds to a feed solution concentration sample of 200 μM used for adsorption studies.

FIG. 9B shows fluorescence peak wavelength vs. cumulative eluent volume, according to aspects of the disclosure. Dark gray points indicate measured fluorescence peak locations. Horizontal dashed lines indicate peak values of solutions containing indicated species (50 μM).

FIG. 10 shows adsorption and desorption cycles of BSA onto a Cibacron blue column using different release agents, according to aspects of the disclosure. The dotted curve represents test cycles with NaSCN (2M) as the release agent, while the solid curve corresponds to test cycles where LA is employed as the release agent for the initial three cycles and NaSCN is utilized as the release agent for the final cycle.

FIG. 11A shows adsorption and desorption cycles of HSA onto a Cibacron blue column using linoleic acid of different concentration, according to aspects of the disclosure.

FIG. 11B shows release peak height and calculated reversible adsorption capacity versus concentration of linoleic acid solution, according to aspects of the disclosure.

FIG. 12 shows an example schematic of a detoxification process of human albumin with designed surface chemistry and biocompatible release agent, according to aspects of the disclosure.

FIG. 13 shows a flowchart of an example method for removal of PBUTs from plasma, according to aspects of the disclosure.

FIG. 14 shows a side view of a dialyzer as an example dialysis cartridge, according to aspects of the disclosure.

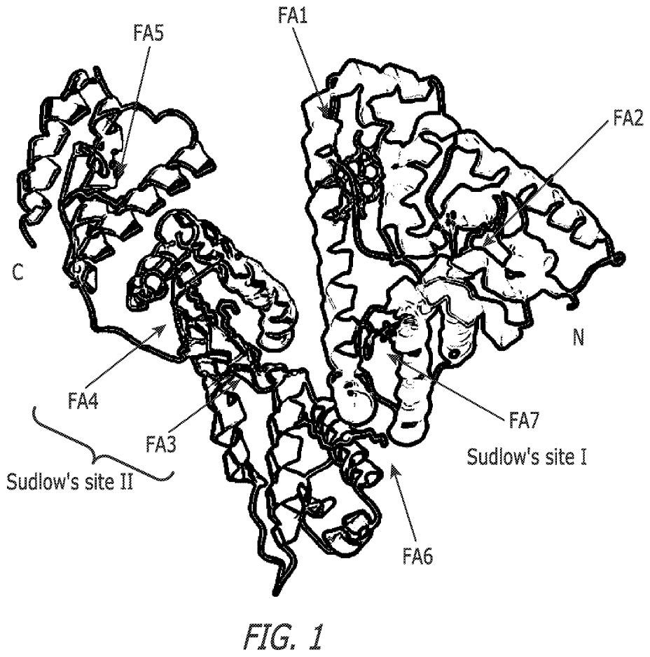

### DETAILED DESCRIPTION

In a general aspect, the disclosure provides dialysis systems, methods, and compositions for removal of PBUTs from a fluid, such as whole blood or plasma of a dialysis patient. The disclosed approaches involve immobilization of toxin-protein complexes to a substrate, removal of toxins from the immobilized toxin-protein complexes to produce clean immobilized proteins, and release of the clean immobilized proteins from the substrate for return of the clean proteins to the dialysis patient. Since the clean proteins are released from the substrate with one or more biocompatible release agents, eluate that includes the clean proteins can be reintroduced to blood of the patient without loss of bioactivity of the proteins, for example, after a small molecule dialysis step, and without risk of adverse health events due to any amount of the release agents in the eluate. The disclosed approaches are economically efficient and enable reduction or elimination of PBUTs during dialysis, thereby reducing the risk of health complications due to the presence of PBUTs in the blood of dialysis patients and increasing survivability of end-stage renal disease (ESRD).

### Dialysis Systems, Compositions, and Methods

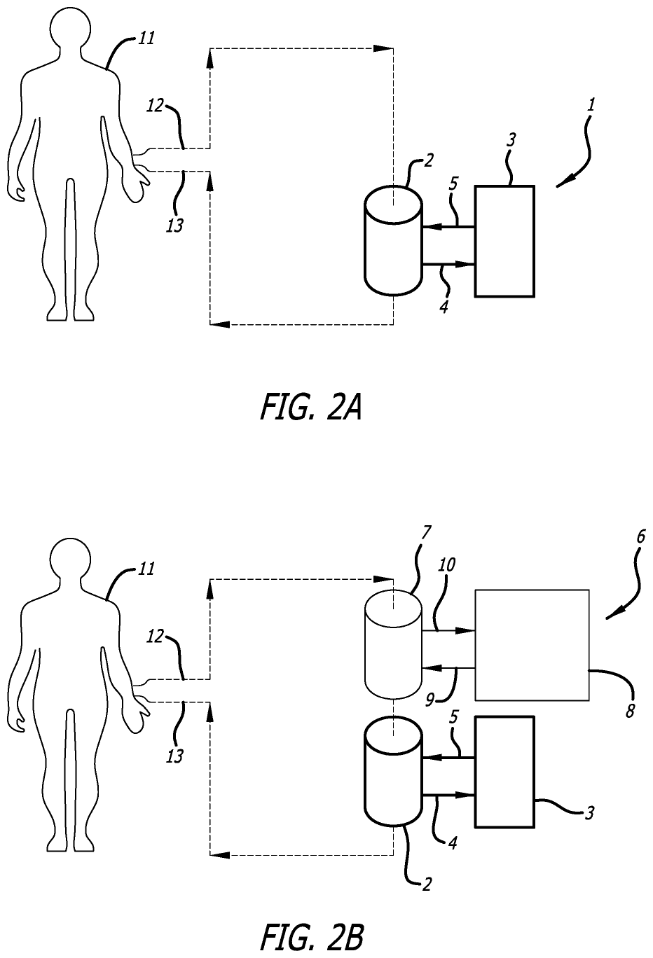

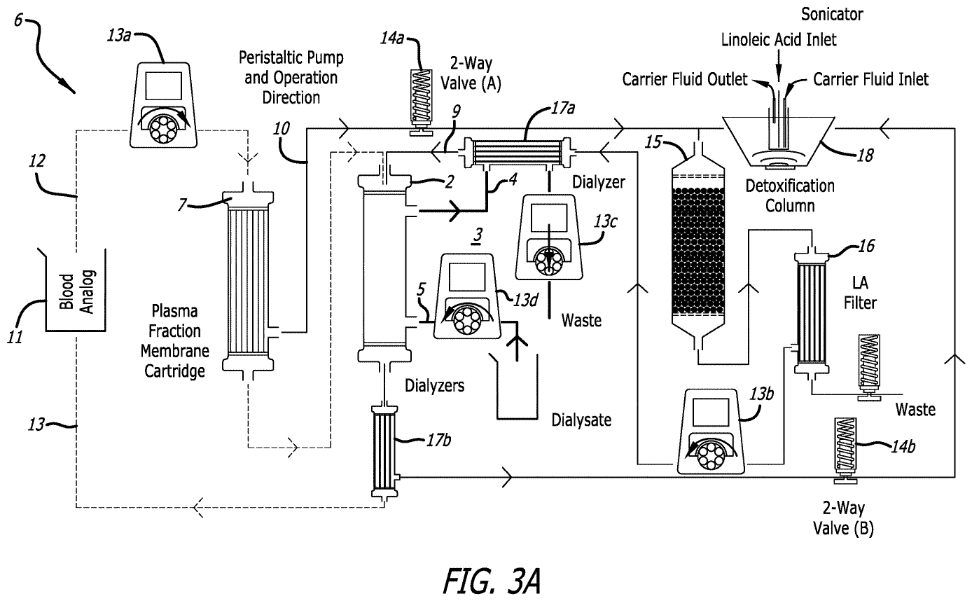

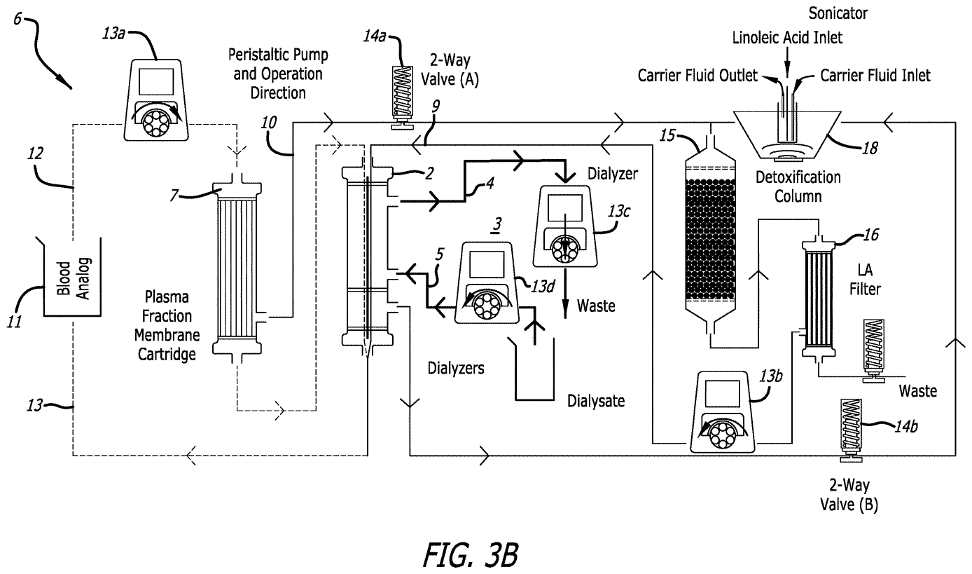

FIGS. 2B, 3A, and 3B show example dialysis systems 6, comprising an immobilized ligand that is configured to bind, e.g., competitively bind, a toxin binding site of a protein and displace a toxin from the toxin binding site of the protein into a first eluate. The immobilized ligand can be bound to a resin of a detoxification column 15, which is a component of a toxin removal loop 8. The dialysis system 6 is further configured for return of the toxin-free protein to blood of a subject.

An existing dialysis system 1, shown at FIG. 2A, includes a dialysis loop 3 that is operably connected with a dialysis cartridge 2. During operation, blood is pumped from subject 11 through inlet tubing 12 to dialysis cartridge 2. The blood is dialyzed with dialysate, across one or more dialysis membranes, through an exchange that includes fresh dialysate tubing 5, which sends fresh dialysate from the dialysis loop 3 to the dialysis cartridge 2, and spent dialysate tubing 4, which returns spent dialysate from the dialysis cartridge 2 to the dialysis loop 3 and/or to waste. Dialyzed blood returns to subject 11 by way of outlet tubing 13. Conventional small molecule dialysis using such an existing system is not effective at removing PBUTs from blood, let alone removing PBUTs and returning cleaned, toxin-free proteins to the subject 11. With such a system, PBUTs accumulate within dialysis patients, which can lead to health complications.

As shown at FIG. 2B, an example toxin removal dialysis system 6 of the disclosure includes a toxin removal loop 8 that is operably connected with a plasma fraction membrane cartridge 7. Toxin removal loop 8 can be installed upstream of the dialysis loop 3, as shown, such that eluate from the toxin removal loop 8 is input to the dialysis loop 3. The plasma fraction membrane cartridge 7 directs at least a portion of plasma of whole blood from the patient to the toxin removal loop 8 by way of plasma inlet tubing 10. PBUTs are removed from the plasma by the toxin removal loop 8, and cleaned plasma including toxin-reduced or toxin-free proteins, flows to dialysis cartridge 2 by way of plasma outlet tubing 9. The toxin-removed plasma is then dialyzed, either as an individual stream within the dialysis cartridge 2 (separate from a whole blood stream), or in a stream that includes the toxin-removed plasma along with the whole blood in combination. Dialyzed whole blood, including toxin-reduced or toxin-free proteins, is then returned to the patient 11 by way of outlet tubing 13.

In embodiments, the toxin removal loop 8 can be installed as an after-market addition to an existing dialysis system (e.g., system 1 of FIG. 2A) to form a toxin removal dialysis system 6 of the disclosure. However, in other embodiments, the toxin removal loop 8 and the dialysis loop 3 are components of a single system, with both elements incorporated into the system during manufacture. In this manner, the toxin removal loop 8 can be included in any dialysis system design or after-market addition, according to need.

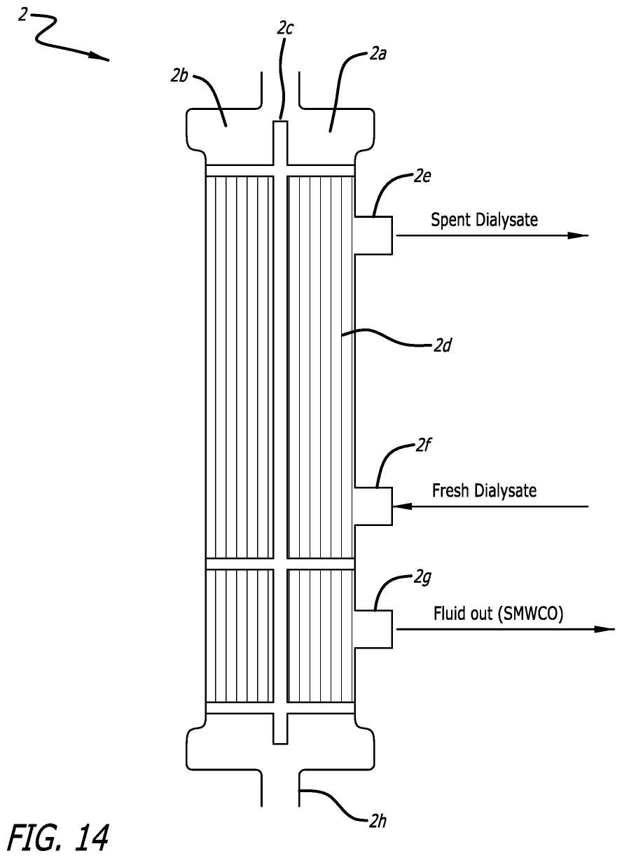

In various aspects, the dialysis system is a kidney dialysis system, a hemofiltration system, a hemodialysis system, a hemodiafiltration system, or another type of dialysis system. As shown at FIG. 3A, a first example toxin removal dialysis system 6 includes combined dialysis of a stream that includes post-toxin removal plasma and whole blood, via dialysis cartridge 2, as well as use of a small molecular weight (SMW) cutoff 17b for recirculation of patient plasma fluids having only small molecular weight compounds through the adsorbent system (i.e., toward valve 14b). However, an alternate configuration is shown at FIG. 3B, wherein a second example toxin removal dialysis system 6 includes separated dialysis of post-toxin removal plasma and whole blood, via modified dialysis cartridge 2. An example of such a modification, as shown by way of example at FIG. 14, is to design the top inlet of a hemodialyzer 2 to be compartmentalized, via a compartment barrier 2c, to the exposed hollow fiber inlets, allowing two parallel streams (e.g., blood at 2b and released PBUT toxin stream at 2a) through the hollow fibers 2d and in contact with dialysate on the outside of the hollow fibers 2d. This design allows for removal of 17a (of FIG. 3A) as a separate cartridge. As shown at FIGS. 3B and 14, near the bottom of dialyzer 2, the outer volume of the cartridge can be compartmentalized with hollow fibers passing through a polymer/resin barrier to produce a second outlet 2g to extract SMW fluids, thereby removing the need for filter 17b, as shown at FIG. 3A. This flexibility in design enables compatibility with existing dialysis cartridges without necessarily needing a dedicated cartridge for the dialysis system 6, and in this manner, the toxin removal loop 8 can be retrofitted or installed as an after-market addition to an existing dialysis system, or alternatively, can be incorporated into the design of a dedicated dialysis system 6, as shown by way of example at FIGS. 3B and 14.

As shown at FIGS. 3A and 3B, the dialysis system 6 comprises a plasma fraction membrane cartridge 7, positioned upstream of a protein-bind column 15, that is configured to divert at least a portion of plasma of whole blood of a subject 11 into the plasma fluid stream and the toxin removal loop through plasma inlet tubing 10.

The example dialysis system 6 includes three loops distinguished by dotted lines, heavy solid lines, and light solid lines: the blood circulating loop, the dialysate loop, and the toxin removal loop, respectively. In embodiments, the dialysate and toxin removal loops can resemble conventional dialyzer devices, with the exception of the plasma fraction membrane cartridge. In embodiments, the toxin removal loop includes the detoxification column 15, which can be a Cibacron blue column, configured for processing albumin and/or other proteins carrying PBUTs.

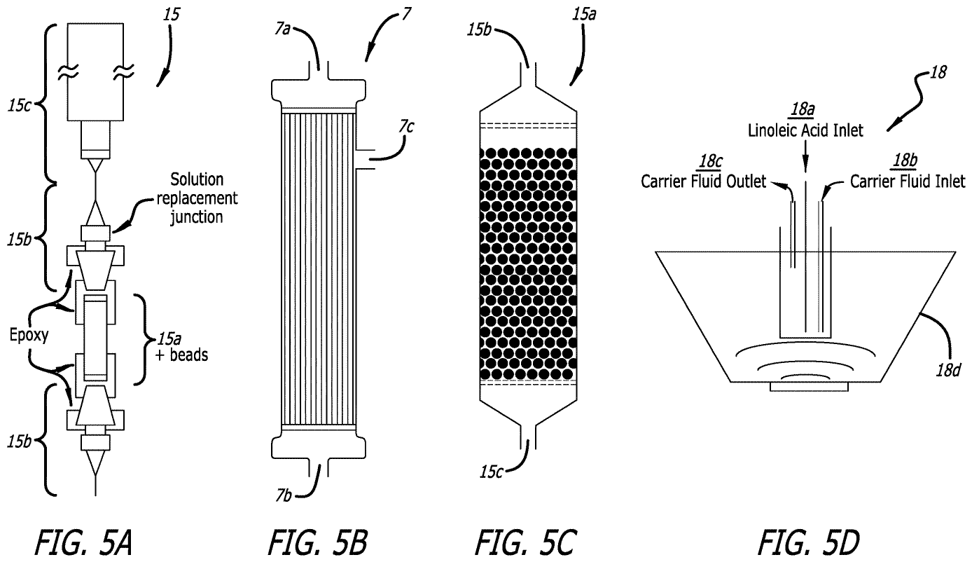

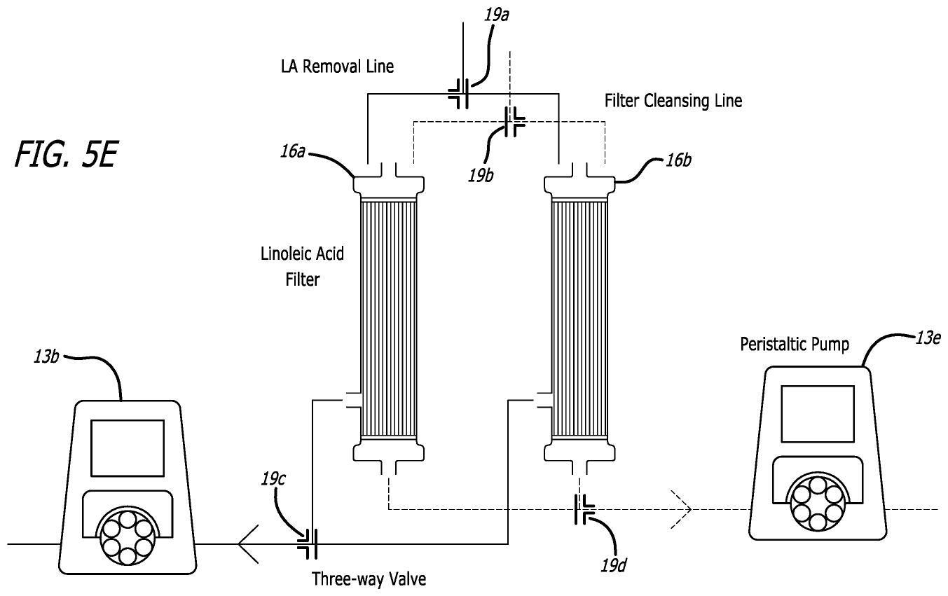

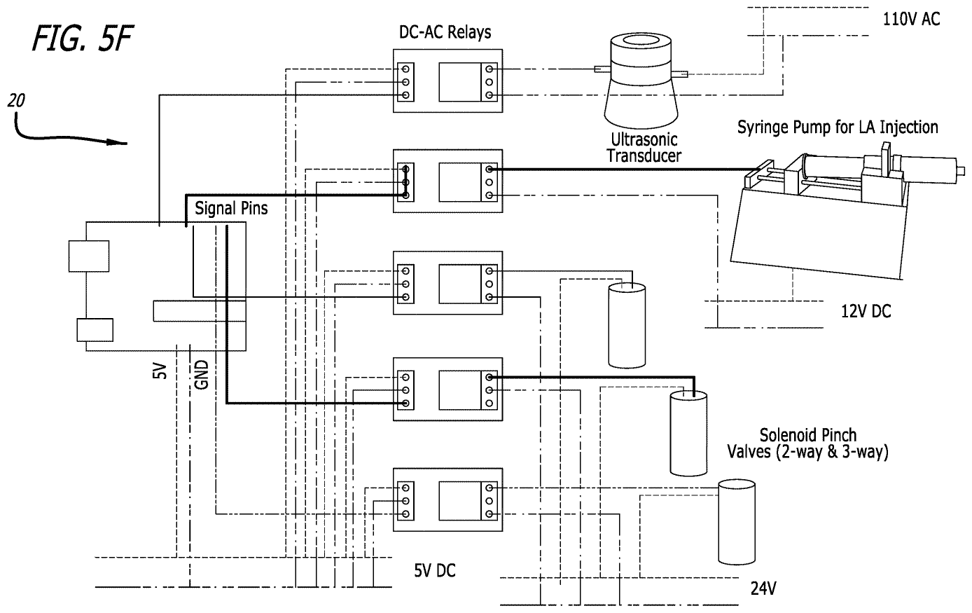

In embodiments, coordination between the various components of the dialysis system 6 can be managed by a preprogrammed Arduino microcontroller unit (MCU), as illustrated by way of example at FIG. 5F. Solenoid pinch valves (14a, 14b of FIGS. 3A and 3B) can be employed to control the flow path of plasma fractionated from the blood loop, while peristaltic pumps (13a, 13b, 13c, 13d of FIGS. 3A and 3B) operated at a constant speed. Other components of the dialysis system 6 can be controlled through power regulation as well.

Operation of the dialysis systems 6 of FIGS. 3A and 3B is divided into two modes: adsorption and desorption of protein or albumin. During an adsorption session, two-way valve 14a remains open while valve 14b is closed. Plasma is then extracted from the blood loop (dotted line) at a rate of 40 ml/min. Albumin or other protein present in the extracted plasma is adsorbed onto an immobilized ligand of the detoxification column 15, while its cargo, indoxyl sulfate (IS) or other toxin, is dissociated and passed into a first eluate. The unbound IS or toxin can be subsequently dialyzed out using dialyzers (e.g., dialysis cartridge 2, dialysis cartridge 17a of FIGS. 3A and 3B).

During the desorption session, valve 14a is closed, and valve 14b is opened. A portion of the blood flow is fractionated and extracted through a dialyzer at the same flow rate of 40 ml/min. The dialyzers do not permit protein permeance. A protein-free carrier fluid delivers a biocompatible release agent, such as linoleic acid (LA), to the albumin-bound detoxification column 15. The dissociation of the protein/immobilized ligand (e.g., albumin/Cibacron blue), caused by the LA solution, passes the protein or albumin from the column into a second eluate. Some, most, or all of the LA in the second eluate can be removed with an LA filter 16. At the LA filter 16, toxin-free (or cleaned) protein or albumin is allowed to transport across the membrane and is sequentially returned to the blood loop, while LA micelles are trapped in the LA filter 16. By cycling between the adsorption and desorption cycles, both free and protein-bound toxins are removed from the blood loop.

While example toxins include indoxyl sulfate (IS), the toxin removal dialysis systems of the disclosure can be used for removal of a wide array of protein-bound toxins, including but not limited to a uremic toxin, a toxin comprising an aromatic-anion structure, p-cresol sulfate, hippuric acid, bilirubin, an indole derivative, IS, p-cresyl sulfate, any derivative thereof, or any combination thereof. In addition, while any suitable immobilized ligand can be used for capture of toxin-protein complexes without departing from the scope and spirit of the disclosure, in embodiments, the immobilized ligand comprises Cibacron Blue (CB). Direct Blue 71, tryptophan with N-terminus functionalized, tryptophan with C-terminus functionalized, octadecane, decane, capric acid, octane, hexane, any derivative thereof, or any combination thereof. Furthermore, while any suitable biocompatible release agent can be used for release of protein or albumin from the detoxification column 15, in example embodiments, the biocompatible release agent comprises a fatty acid, LA, a fatty acid mix, an inhomogeneous emulsion comprising LA, oleic acid, palmitic acid, any derivative thereof, or any combination thereof.

In embodiments, the dialysis system 6 further comprises a dialyzer 2 that is configured for small molecule hemodialysis of the first eluate, the second eluate, or both, into dialysate loop 3. The dialyzer 2, which can be a hemodialyzer, can be configured for separated dialysis of a first stream that includes post-toxin removal plasma and a second stream that includes whole blood, such that the first stream and the second stream are combined after flow through the dialyzer 2, as shown by way of example at FIG. 3B.

FIG. 14 shows a side view of a dialyzer as an example dialysis cartridge 2, which can be a hollow fiber cartridge, in embodiments. In the shown embodiment, dialysis cartridge 2 includes a first compartment 2a and a second compartment 2b that are separated by a compartment barrier 2c; the first compartment 2a can receive the plasma stream therein and the second compartment 2b can receive the whole blood stream therein, or vice versa, and each can be defined by a certain percentage for the stream, such as 30% of the cartridge volume used for the plasma stream and 70% of the cartridge volume used for the whole blood stream, for example. Since the cartridge can be a hollow fiber cartridge, the partitioned streams pass through separated hollow fibers 2d and do not interact with each other while passing through the dialysis cartridge 2, but are individually dialyzed by way of dialysate that passes through a dialysate inlet 2f (fresh dialysate) and a dialysate outlet 2e (spent dialysate). In embodiments, the dialysis cartridge 2 can further comprise a small molecular weight (SMW) cutoff filter that divides the external fluid (outside the hollow fibers), shown as a horizontal double line approximately two-thirds down from the top of the dialysis cartridge 2. In this manner, a fluid outlet 2g can be included for use of the SMW cutoff filter for recycling small molecular weight compounds back through the dialysis system. In this example design, including as shown at FIG. 3B, the additional SMW filter 17b (of FIG. 3A) can be optional in at least some embodiments of the dialysis system, due at least in part to inclusion of this or a similar function in the example dialysis cartridge 2 of FIG. 14, for example, by way of the SMW cutoff filter.

FIGS. 5B and 5D show an example assembled plasma/linoleic acid (LA) filter 7 and an example sonicator 18 with carrier flow and LA flow, respectively. The plasma filter and linoleic acid filter 7, which includes an inlet 7a, outlet 7b, and outlet 7c, can be assembled using a set amount of PlasmaPhan hollow fiber membranes (3M®). The membranes can be potted with epoxy in a designed barrel. Once the epoxy is properly cured, the excess length of the hollow fiber can be trimmed using a scalpel to expose the potted hollow fibers. Subsequently, a Luer lock connector can be attached to the filter cartridge. After the assembly process, the hollow fibers can be wetted with ethanol and rinsed with a generous amount of water. An adequate quantity of dialysate can be employed to flush the cartridge before its use. The effectiveness of the linoleic acid filter can be evaluated by intentionally obstructing the filter with a linoleic acid suspension (8 μl/10 ml buffer), while observing changes in both the volume of the feed solution and the clarity of the eluent. To investigate the regeneration of the filter, various solutions can be employed to flush the filter in different directions. For linoleic acid dispersion, a sonicator 18 can be constructed using a custom-made power supply and frequency generator. The sonication bath can be comprised of a ceramic plate transducer (43 kHz, 35W) attached beneath a stainless-steel bowl using cyanoacrylate glue. A 20 ml glass vial can be securely positioned in the center of a sonication bath 18d with a tubing arrangement, including LA inlet 18a, carrier fluid inlet 18b, and carrier fluid outlet 18c, as outlined at FIG. 5D.

Linoleic acid is inherently insoluble in water, and linoleic acid solutions can be considered to be suspensions of LA micelles. An increased concentration of LA (8 μl/10 ml), in contrast to a physiological concentration (1.8 μl/10 ml), necessitates removal of at least excess levels of the LA from the second eluate. Given that the size of LA micelles far exceeds 100 nm level, the hollow fiber membranes, specifically PlasmaPhan and MicroPES, with pore sizes not exceeding 500 nm, are capable of capturing a substantial portion of LA micelles. Through multiple experiments using a dead-end filter configuration, it was determined that 15 strands of PlasmaPhan (and MicroPES) hollow fiber membrane can filter 30 ml of an 8 μl/10 ml LA solution before becoming completely obstructed. This translates roughly to a LA removal capability of 2 ml of LA solution per hollow fiber. Therefore, considering the designed prototype operation of a 4-hour session, during which half of the time is allocated for LA desorption, the volume of LA solution to be processed would be 4800 ml, and the required amount of hollow fiber membrane would be 2400, unless the LA filter can be regenerated during operation. While industrial production of hollow fiber cartridges easily incorporates thousands of hollow fibers, creating a filter cartridge with high fiber count presents challenges in laboratory settings. Consequently, the potential for regenerating the LA filter was explored. The intentionally blocked LA filter was found to impede the flow of liquid in any direction. However, when the flow was reversed (opposite to the filtration direction), it loosens the blockage to some extent. At this point, it was discovered that the addition of alcohols, IPA and ethanol, effectively alleviates the obstruction, and the reversed flow washes out fiber-shaped precipitates. Under this circumstance, the blockage was attributed to the accumulation of such precipitates. Without wishing to be bound by any particular theory, the precipitate can potentially be the result of linoleic acid oxidation in ambient air, although this has not been confirmed through characterization. To allow easier access to remove the LA precipitate, the design of the blood fractionation filter was adopted as the linoleic acid (LA) filter. Illustrated at FIG. 5B, the filter enables cross-membrane flow when one end (outlet 7b) of the filter cartridge is closed and allows axial flow when both ends of the filter cartridge are open (inlet 7a, outlet 7b). This mechanism facilitates LA precipitate removal by the axial flow (outlet 7c). More fiber-shaped precipitate was observed during axial rinsing of ethanol-water in sequence through the hollow fibers. This resulted in the relief of blockage, allowing unimpeded axial flow. However, when LA solution was subsequently filtered through the unblocked filter, it became blocked again after passing only 18 ml of LA solution, whereas the initial blockage required 30 ml of LA solution. Subsequent unblocking and LA filtering revealed an earlier blockage at 13 ml of LA solution but stabilized for 5 unblock-block cycles. Without wishing to be bound by any particular theory, this phenomenon can potentially be a consequence of liquid passing through the opened fibers while the blocked fibers remained obstructed.

For a dialysis system operation spanning 4 hours with regeneration every 20 minutes, each cartridge would be required to process 400 ml of linoleic acid solution, hence necessitating 462 hollow fibers of PlasmaPhan or MicroPES. To prevent the loss of filter capacity, as discussed above, it's important not to saturate the filtering capacity. Consequently, the LA filter cartridge can be designed with 600 hollow fibers. Given the need for continuous prototype operation, a parallel operation-cleansing line design can be implemented, as illustrated in at FIG. 5E. In this configuration, one LA filter cartridge would be in operation while the other undergoes cleansing by axial flow. With the example LA filter configuration for operation and inline cleansing, this arrangement includes two LA filters (16a, 16b), LA removal line, filter cleansing line, and three-way valves (19c, 19d) operated by peristaltic pumps (13b, 13e).

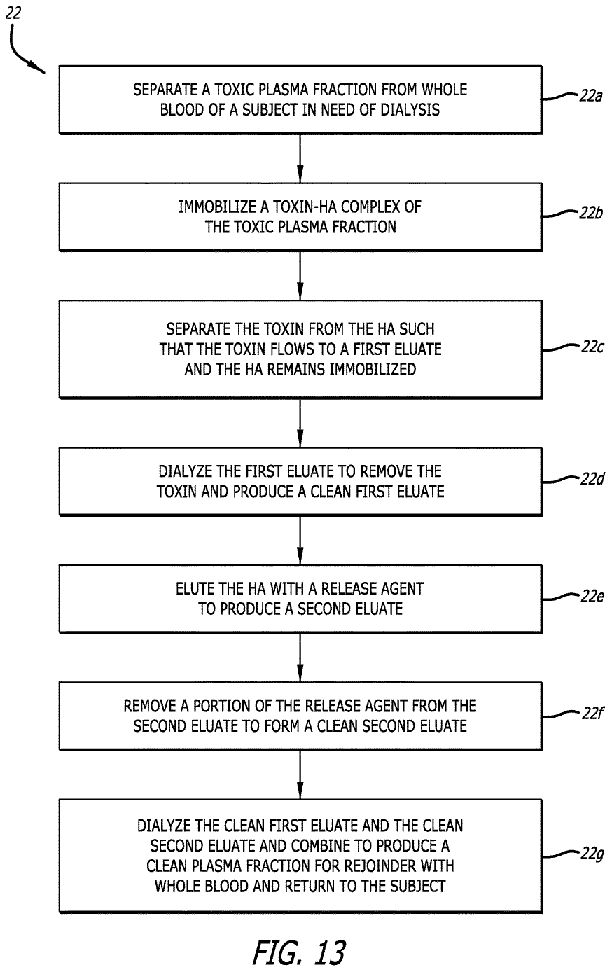

In an aspect, the disclosure provides hemodialysis methods, as shown by way of example at FIG. 13. A hemodialysis method 22 includes, at step 22a, separating a toxic plasma fraction from whole blood of a subject in need of dialysis; at step 22b, binding a toxin-protein complex of the toxic plasma fraction to an immobilized ligand; at step 22c, separating a toxin from the toxin-protein complex such that the toxin flows to a first eluate and a protein of the toxin-protein complex remains bound to the immobilized ligand; at step 22d, dialyzing the first eluate to remove the toxin and produce a clean first eluate; at step 22e, eluting the protein from the immobilized ligand with a biocompatible release agent such that the protein flows to a second eluate; at step 22f, removing at least a portion of the biocompatible release agent from the second eluate to form a clean second eluate; at step 22g, dialyzing and combining the clean first eluate and the clean second eluate to produce a clean plasma fraction; and returning at least a portion of the clean plasma fraction to blood of the subject.

A composition produced by a system or method of the disclosure can comprise a clean plasma fraction. The clean plasma fraction can be introduced to a subject as part of a method for treating end stage renal disease (ESRD).

### Fluid Regeneration Systems and Liquid Dialysis Circuits

In embodiments, a dialysis system of the disclosure incorporates a fluid regeneration system as described in U.S. Pat. No. 10,973,971, which is expressly incorporated herein by reference in its entirety. The fluid regeneration system is configured for urea removal from a dialysate, and as part of a dialysis system of the present disclosure, can be used for dialysis, including kidney dialysis, hemodialysis, hemofiltration, hemodiafiltration, removal of impurities, etc. In such a system, a photo-chemical oxidation (also referred to as “dialysis-fluid regeneration” or “urea treatment”) removes urea from dialysate. A dialysis system fluid regeneration system can include a nanostructured anode, a source of light configured to illuminate the anode, and a cathode that is oxygen permeable. The nanostructures can be TiO₂ nanowires that are hydrothermally grown. The source of light can be provided by an array of light-emitting diodes (LEDs). The oxygen permeable or air permeable cathode can be a platinum-coated (Pt-coated) cloth or paper. Such a system can be sized down enough to become wearable and/or portable, for example. Wearable dialysis devices not only achieve continuous dialysis, but also help reduce clinic related treatment costs and improve quality of life through enhanced mobility. Use of such a system can include a method for regenerating a dialysis fluid, comprising flowing the dialysis fluid between an anode that comprises a plurality of nanostructures and a cathode of a dialysis system, illuminating the anode with a source of light, flowing oxygen through the cathode toward the dialysis fluid, and converting urea in the dialysis fluid into CO₂, N₂ and H₂O thereby regenerating the dialysis fluid.

In embodiments, a dialysis system of the disclosure incorporates a liquid dialysis circuit as described in the International Patent Application Number PCT/US2022/023305, filed Apr. 4, 2022, and published as International Publication Number WO 2022/216604, on Oct. 13, 2022, which is expressly incorporated herein by reference in its entirety. As part of a dialysis system of the present disclosure, the liquid dialysis circuit can utilize osmotic membranes or other membrane types that are highly selective to urea transport to achieve diffusional flux of urea in a forward osmosis geometry. When combined with an oxidation unit, this protected geometry can have a high selective flux of urea from the spent dialysate side to the urea removal side. This enables balance of fluid (water) levels in patients by forward osmotic flow by controlling water evaporation rate through a vapor permeable membrane, protection of patient dialysate/blood loop from oxidation by-products, and an ability to optimize oxidation system performance (pH, ionic strength, other electrolytes, etc.) that would be otherwise incompatible with blood contact. Such a liquid dialysis circuit can comprise a dialysate loop, separated from a patient blood circuit by a dialysis membrane, and a toxin-removal loop separated by a toxin-selective membrane configured to selectively pass the toxin from the dialysate loop to the toxin-removal loop, for example.

TABLE 1
K_{d} of HA on SPR sensor modified with different ligands.

| Surface Ligand | K_{d} (μM) |
|---|---|
| Cibacron Blue | 3.0 |
| Direct Blue 71 | 1.1 |
| Tryptophan | 0.9 |
| Octadecane (C18; Alkane) | 2.0 |
| Decane (C10; Alkane) | 1.1 |
| Capric Acid (C10; COOH) | 0.9 |
| Hexane (C6; Alkane) | 1.6 |

TABLE 2
Summary of albumin absorption and desorption on SPR chips as a function
of surface terminus functionalization and release agent.

| | SPR | Release percentage (highest observed; %) | | | | | | | |
|---|---|---|---|---|---|---|---|---|---|
| | angle shift | | | | | SDS | LA | | lbp |
| Surface | after HA | NaSCN | NaCl | Na₂SO₄ | pH | (35 | (0.6 | FA | (0.1 |
| ligand | binding (°) | (2M) | (2M) | (2M) | 3.6 | mM) | mM) | mix | mM) |
| Cibacron | ~0.25 | 78.0 | 34 | 12.7 | 0 | 93.0 | 44.1 | 49.5 | 0 |
| Blue (CB) | | | | | | | | | |
| Direct Blue | ~0.19 | 0 | — | — | | 0 | — | — | — |
| 71 | | | | | | | | | |
| Tryptophan | ~0.17 | 15.0 | — | — | 0 | 0 | — | — | — |
| (NH₂ term) | | | | | | | | | |
| Tryptophan | ~0.19 | 0 | — | — | 0 | — | — | — | — |
| (COOH | | | | | | | | | |
| term) | | | | | | | | | |
| Octadecane | ~0.24 | 42.0 | — | — | — | 0 | — | — | — |
| (C18: | | | | | | | | | |
| Alkane) | | | | | | | | | |
| Decane | ~0.15 | 0 | — | — | — | 38.1 | — | — | — |
| (C10; | | | | | | | | | |
| Alkane) | | | | | | | | | |
| Capric Acid | ~0.18 | 65.0 | 37.1 | 0 | 18.7 | 72.5 | 52.3 | 36.1 | |
| (C10; | | | | | | | | | |
| COOH) | | | | | | | | | |
| Octane (C8; | ~0.11 | 51.4 | — | — | — | 29.5 | — | — | — |
| Alkane) | | | | | | | | | |
| Hexane | ~0.05 | 0 | — | — | — | — | — | — | — |
| (C6; | | | | | | | | | |
| Alkane) | | | | | | | | | |

TABLE 3
Human serum albumin desorption percent as a function of
release agent on capric acid and Cibacron Blue functionalized
chips. Values shown are in percentage with respect to
a monolayer protein binding signal strength.

| | Release Agent | | |
|---|---|---|---|
| | | Fatty Acid | |
| Surface | Linoleic Acid | Mix | NaSCN |
| Capric Acid | 27.5 ± 15.15% | 38.9 ± 9.9% | 49.5 ± 20.4% |
| | (n = 14) | (n = 15) | (n = 20) |
| Cibacron Blue | 31.0 ± 18.9% | 37.4 ± 14.2% | 65.0 ± 12.3% |
| | (n = 12) | (n = 18) | (n = 45) |

### EXAMPLES

### Example 1. Removal of Protein-Bound Uremic Toxins (PBUTs) from Human Albumin (HA)

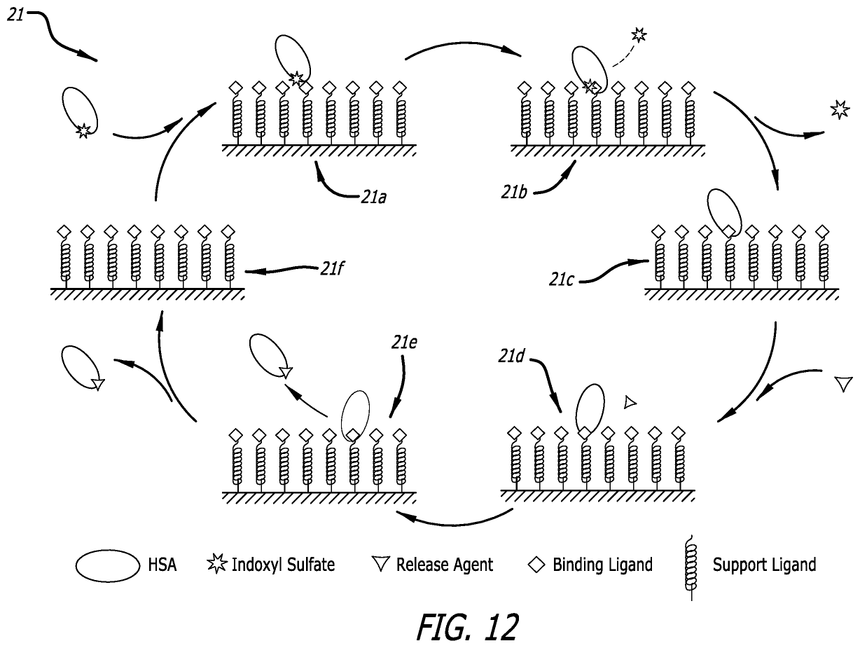

In this example, a 2-step albumin regeneration process with HA-selective binding and release for selective removal of PBUTs from S-II site of HA, as shown at FIG. 12, is demonstrated. A variety of surface chemistries that selectively bind to HA and simultaneously forces the release of IS from S-II sites were tested, and a series of plasma-safe release agents for releasing cleansed HA from the surfaces was studied. Cibacron Blue and physiological fatty acid mixtures were found to be effective binding and release agents. The albumin regeneration chemistry was then applied to an agarose bead support showing binding and release, which can be scaled to regenerate 250 g of HA daily while using only 155 ml of agarose/CB resin with 2-minute cycles.

As shown at FIG. 12, an example albumin regeneration process 21 includes immobilization of an HSA-IS complex to a support ligand 21a, displacement/removal of IS from HSA 21b to produce clean immobilized HSA 21c, introduction of a release agent 21d which releases the HSA from the ligand, to produce cleaned and released HSA 21e and a recharged support ligand 21f. The recharged support ligand is available for use in a further cycle of the albumin regeneration process 21.

This example relates to improved compositions, systems, and methods for release of PBUTs from HA followed by release of cleaned HA back into the blood stream with a physiologically safe release agent for use in dialysis, including a two-step albumin regeneration process with HA-selective binding and release for selective removal of PBUTs from an S-II site of HA. A variety of surface chemistries were tested that selectively bind to HA and simultaneously force the release of IS from S-II sites. Then, a series of plasma-safe release agents for releasing cleansed HA from the surfaces was studied. Cibacron Blue and physiological fatty acid mixtures were found to be effective binding and release agents. The albumin regeneration chemistry was then applied to agarose bead support showing binding and release which can be scaled to regenerate 250 g of HA daily while using only 155 mL of agarose/CB resin with 2-minute cycles.

A series of covalently bonded surface functionalizations, ranging from long-chain alkanes, lipids, and charged aromatics, were studied for HA binding via surface plasmon resonance (SPR). Desorption coefficients (K_{d}) ranged from 0.9 to 3 μM and were comparable to HA binding to indoxyl sulfate (IS) toxin. A series of release agents were studied, ranging from physiologically toxic NaSCN, sodium dodecyl sulfate (SDS), to physiologically manageable NaCl, HCl and fatty acids. Herein it is disclosed that plasma compatible fatty acids (e.g., linoleic acid (LA), and fatty acid mixtures of physiological ratios) can release cleaned HA with about 30-40% surface coverage. The example process was scaled to commercially available Cibacron blue functionalized agarose beads commonly used for HA purification by chromatography. Binding capacities of 0.35 μmol HA/mL beads and release amounts of 0.23 μmol HA/mL beads are shown using the plasma compatible linoleic acid. Fluorescence studies of the released HA eluents showed the HA-IS complex had been stripped of the protein bound toxin. IS. During a conventional 4-hr dialysis session with 2-minute binding release cycles, only 155 mL of CB-agarose beads would be needed to process the 250 g of HA in a typical patient, enabling a practical treatment strategy.

### Materials and Methods

SPR sensor chip cleaning: As-received SPR sensor chips were rinsed with de-ionized (DI) water and ethanol followed by Ar drying. Dried chips were treated with oxygen plasma in a plasma cleaner (Model PDC-001-HP, Harrick Plasma) and stored under Ar.

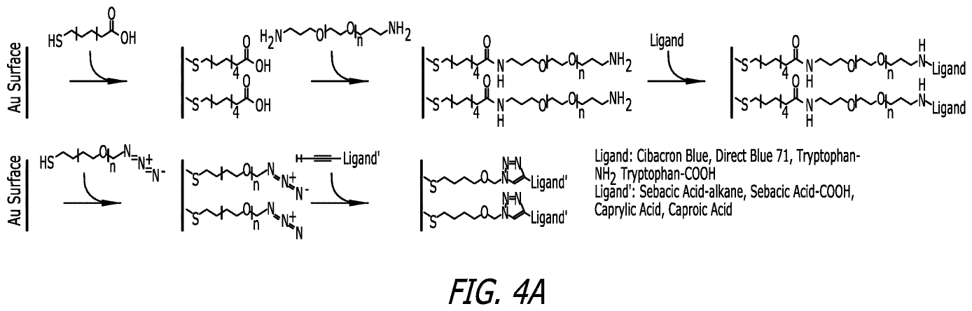

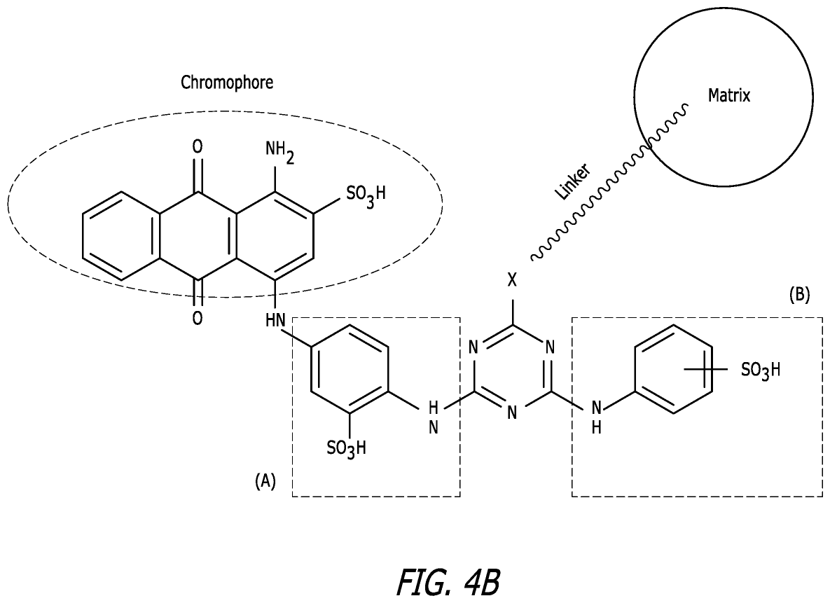

Ligand grafting onto SPR sensor chips (see also FIG. 4A): MUDA grafting: 10 mM 11-mercaptoundecanoic acid (MUDA) was dissolved in Ar-purged dry ethanol. Clean SPR chips were immersed in prepared solution, and then set on an orbital shaker overnight under Ar protection, then twice rinsed and sonicated in ethanol followed by drying and storage in an Ar atmosphere. H₂N-PEG-NH₂ grafting: 19.8 mg bis(3-aminopropyl) terminated PEG (Mn˜1500), 4 mg 1-(3-dimethylaminopropyl)-3-ethylcarbodiimide hydrochloride (EDC), and 3 mg N-hydroxysuccinimide (NHS) were dissolved via sonication in 20 mL dry DMF. SPR chips were immersed in this solution and left on a shaker overnight in an Ar atmosphere. Cibacron Blue grafting (see also FIG. 4B): In 70 mL of Milli-Q water, 51.3 mg Cibacron Blue and 2.336 g NaCl were dissolved with aid of bath sonication. This solution was heated to 60° C. while stirring and tuned to pH 10-11 with NaOH for reaction for 4 hr. Concentration change due to evaporation was compensated with DI water. Functionalized chips were rinsed with water and ethanol then stored in an Ar atmosphere. Decanoic acid grafting: PEG-modified SPR chips were treated with 20 mL DMF solution with 8.9 mg sebacic acid bis(N-succinimidyl) ester overnight in an Ar atmosphere on an orbital shaker. Yielded chips were rinsed with DMF and ethanol followed by drying and storage in Ar before use. Tryptophan grafting: Tryptophan was grafted onto PEG-modified SPR chips by two different mechanisms to produce different spatial orientations (C or N terminus). A coupling solution was made by dissolving 3.1 mg EDC, 2.3 mg NHS, and 4.1 mg tryptophan in 20 mL dry DMF. i) Tryptophan was coupled by its —COOH group with the —NH₂ groups on a PEG-modified chip surface by immersing chips in the coupling solution overnight on an orbital shaker. ii) Tryptophan was attached by its —NH₂ group to the chip surface. This process includes an additional disuccinimidyl carbonate (DSC) activation step, 30 min. in 51.2 mg DSC 20 mL DMF solution, prior to the EDC coupling mentioned above. Tryptophan-grafted chips were rinsed with DMF and ethanol and stored in inert gas. Direct Blue 71 grafting: DSC activated PEG-modified SPR chips were treated with DB71 solution made of 193.2 mg Direct Blue 71 in 20 mL dry DMF. After grafting, the SPR chips were rinsed with DMF and ethanol and then dried and stored in an Ar atmosphere. Stearic Acid grafting: Thiol PEG (M.W. 3400 Da) with a stearic acid terminus was purchased and used as-is. SPR chips were immersed in solutions of 25.5 mg stearic acid thiol PEG in 15 mL Ar-purged chloroform overnight on an orbital shaker. The resultant grafted SPR chips were rinsed with chloroform and IPA and then dried and stored in Ar. Thiol azide PEG grafting: Thiol PEG (M.W. 3400 Da) with an azide terminus was purchased and used as-is. Cleaned SPR chips were immersed in a solution of 20 mg thiol-azido-PEG in 20 mL Ar-purged DMF for over 6 hr. The reaction was protected from light and the solution and chips were shaken on an orbital shaker. The resultant SPR chips were rinsed with DMF and stored in Ar. Alkane chain grafting: Alkane chains with different chain lengths, 1-hexyne to 1-dodecyne, were grafted via click chemistry on the azide PEG grafted chips. The desired alkyne, CuSO₄, and ascorbic acid were dissolved in a 1:1:1 molar ratio in a 4:1 v/v DMF:water solvent to make a 1 mM alkyne solution. The thiol azide PEG grafted chips were immersed in such solution overnight in an Ar atmosphere and protected from light. Yielded chips were rinsed with DMF and ethanol prior to drying and storage before use.

SPR tests: A temperature controlled NanoSPR8 Model 481 device with a 4-channel flow cell was used and calibration and warm up were performed (2 hr) prior to tests. Two flow cell channels were set as reference in all tests. Basic solution with surfactant (0.1 M NaOH and 1% SDS). acidic solution (0.1 M HCl), and water were used to clean the testing flow cell and tubing system.

A phosphate buffer solution (PB) for SPR studies was prepared by dissolving 21.48 g Na₂HPO₄ and 3.11 g KH₂PO₄ in 1 L Milli-Q water for SPR tests. With this buffer a variety of solutions were prepared: NaCl. Na₂SO₄ and NaSCN was dissolved in the PB to prepare a 2 M solution. Sodium dodecyl sulfate (SDS) solution of 1 wt % (˜35 mM) was prepared by dissolving SDS in PB with aid of bath sonication. Fatty acid solutions were made by pipetting fatty acid into PB and bath sonicating. Linoleic acid solution (LA) included 1.8 μL of LA and 10 mL PB. Fatty acid mix solution (FA mix) was prepared by dispersing 1.8 μL linoleic acid, 1.05 μL oleic acid and 1.08 μL palmitic acid in 10 mL PB. FA mix was an inhomogeneous emulsion due to the limited solubility of fatty acids even with the aid of bath sonication.

Langmuir Isotherm: HA was dissolved in PB to make solutions with concentration range from 0.1 mg/mL to 2.0 mg/mL for adsorption study. Such HA solutions were pumped through the flow cell in low to high sequence with 20 min. adsorption time for each solution. Between each HA adsorption session, buffer was used to rinse off unbound protein residue for 20 min. PB signal from selected reference channels was used as baseline.

Prior to further analysis, the raw SPR data, absolute maximum absorption angle versus time, was referenced to a baseline reading. A 3-point running average was applied to reduce noise.

The values of SPR signal shift (in degrees) induced by HA solutions of different concentrations were manually identified. For Langmuir adsorption model fitting, the SPR signal shifts were converted to fractional occupancy of adsorption sites (θ) by assuming 0% and 100% site occupancy under 0 mg/mL and 2 mg/mL HA solution treatment, respectively. After plotting the concentration on x-axis and θ on y-axis, the data was fitted to a Langmuir isotherm model with equation

$$θ = \frac{{K}_{α} \times Conc .}{1 + {K}_{a} \times Conc .} .$$

The adsorption constant, Kₐ, was obtained under the best fitting condition. The desorption constant. K_{d}, reported in Table 1, was calculated by K_{d}=1/Kₐ.

Adsorption and desorption cycles: Cyclic tests were performed with functionalized SPR sensor chips and 2 mg/mL HA solutions. The test was carried out in sequence of HA solution, buffer rinse, releasing agent rinse, buffer rinse, in cycles after an initial PB rinse. Three absorption and desorption cycles were performed on each sample for repeatability/stability. The first two cycles used the same release agents of interest and the third cycle used a reference release agent, usually 2 M NaSCN.

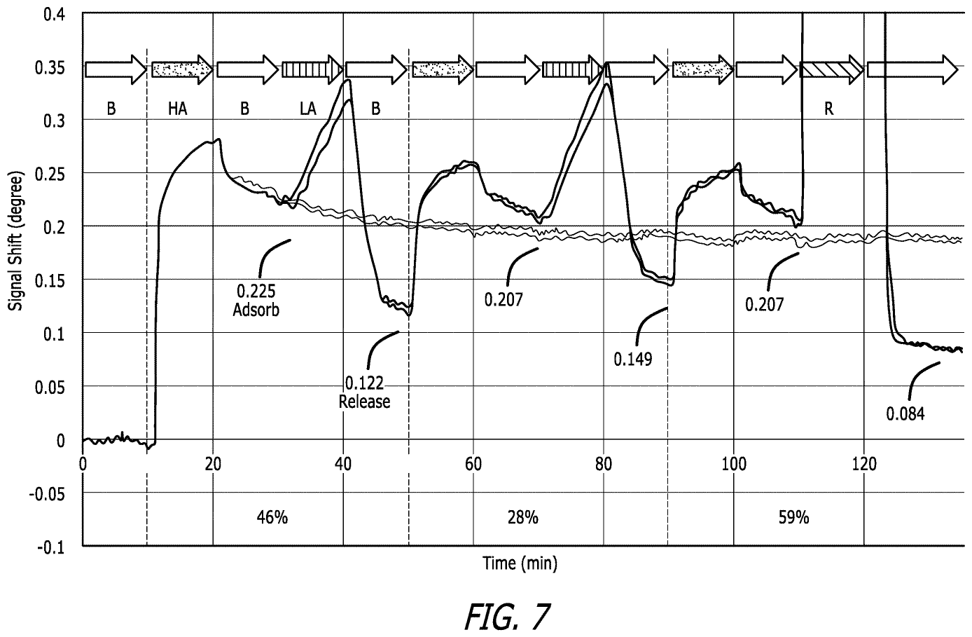

Percentage of HA released by different release agents was calculated by dividing the value difference between the albumin adsorption SPR peak shift value (‘Adsorb’ in FIG. 7) and post-release signal value (‘Release’ in FIG. 7) using

$$Release Percentage = \frac{Ad - Rel}{Ad} \times 100 \% .$$

Adsorption column (set-up): Devcon 5-minute epoxy in Dev-tube (ITW Polymer Adhesives, USA) was used on all epoxied parts of the column and was allowed to cure for an hour before use. Agarose beads functionalized with Cibacron Blue dye and suspended in 20% ethanol and 0.1 M KH₂PO₄ (Blue Sepharose Fast Flow 6, GE Healthcare, USA) was obtained and used as-is.

As shown at FIGS. 5A and 5C, glass tube 15a holds agarose beads functionalized with human albumin binding chemistry Cibacron Blue dye, and a barbed inlet/outlet 15b with Luer lock and needle tip is included to flow a solution in and out of glass tube 15a. An interchangeable solution reservoir feed 15c is included. Specifically, glass tube 15a (inner diameter=4 mm) was cut to 4.1 cm (0.52 mL volume). Two sheets of 10.0 μm pore size Teflon membrane filters (Sterlitech Corp., USA) covered the eluent end of the tube and were sealed around the edge with larger diameter elastic tubing (Tygon SE-200, Saint-Gobain Corp., USA) with excess membrane trimmed by knife. The tubing was secured to the tube via glass-compatible glue (Loctite, Henkel AG & Co. KGaA. Germany) and then epoxied to prevent air leakage. The beads in their suspension fluid (0.739 mL total slurry) were pipetted into the tube on top of the membrane seal and drained to form a bed of beads. The other end of the tube was sealed with membrane filters and tubing in a similar manner. Both ends 15b of the bead bed tube were connected to a Luer lock needle assembly that were epoxied to input tubing to prevent fluid leakage. The solution reservoir 15c included a syringe and a Luer lock connection. The column was suspended vertically and syringe pump (Model NE 300, New Era Pump Systems, Inc., USA) set to 4.0 mL/min with 125-250 μL samples collected at time points with lag time experimentally accounted for. Replacing of solutions occurred by switching out the syringe at the base of the Luer lock needle assembly in 15b, ensuring no air bubbles were introduced.

A phosphate buffer (PB) (pH 7.4) for the adsorption column was prepared to mimic the salt composition of human blood serum. 144.3 mg/L CaCl₂), 4.26 g/L Na₂HPO₄, 612.4 mg/L KH₂PO₄, 85.7 mg/L MgCl₂, and 4.68 g/L NaCl were dissolved in HPLC-grade submicron filtered water under stirring and heat, then filtered through a 10.0 μm membrane filter. NaCl was purchased from EMD Chemicals Inc. (Germany): all other chemicals were obtained from Sigma-Aldrich (USA). NaSCN, indoxyl sulfate, and HA solutions were prepared by simple dissolution in PB. 0.58 mM linoleic acid (LA) solution was prepared by pipetting liquid LA into phosphate buffer and sonicating in 10-minute increments until micelles were formed and evenly distributed, indicated by the solution becoming uniformly cloudy. Solutions of micro BCA protein assay were prepared as instructed in a kit obtained from Thermo Scientific (USA).

Absorption and Fluorescence Measurements: Absorbance and fluorescence measurements were taken with a UV-Vis spectrophotometer (Molecular Devices SpectraMax i3x). Eluent solutions from the absorption column were collected in fluorescence plates (Thermo Scientific Nunc, USA). Fluorescence spectrum scans from 320-410 nm were recorded for each sample with an excitation wavelength of 270 nm and bandwidth of 15 nm. For absorbance measurements, each sample was then transferred and diluted to its ⅛ strength in absorbance plates (Corning, USA). For BCA analysis, absorbance samples were further diluted to 1/533 in a new absorbance plate. A 6-point calibration curve was used and data normalized to known maximum and minimum values. Binding capacities were measured by trapezoidal integration of eluent concentration as a function of time during release/rinse periods. Free volume was calculated from bead volume of 0.38 mL and spherical packing density of 0.64.

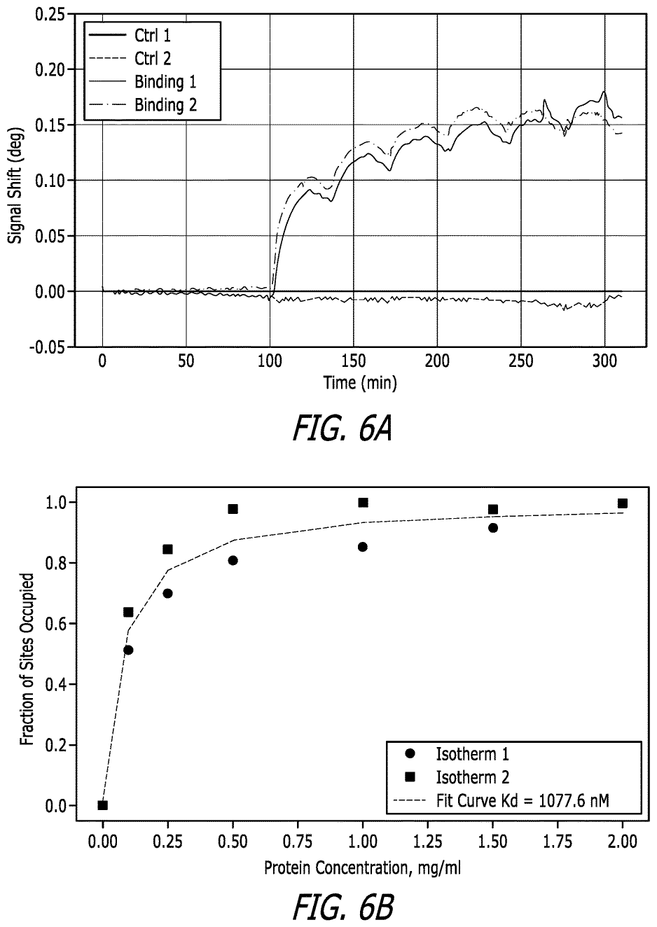

HA selective ligands were grafted onto SPR chips with a three-layer structure: Thiol-gold chemistry, as the bottom layer, immobilized thiol-azido PEG or MUDA (and PEG) onto the chips while PEG provide anti-fouling layer in the middle. HA selective ligands, targeting S-II sites and fatty acid binding sites, were grafted on top of the MUDA or PEG layer. FIG. 6A shows a representative adsorption of HA onto the modified chip surface monitored by SPR signal shift as a function of increased concentration. The binding behavior signal (binding 1 & 2) was fit to the Langmuir isotherm model (isotherm 1 & 2) and plotted in FIG. 6B with adsorption coefficient calculated accordingly.

The K_{d}s of various surfaces show in a narrow range of 0.9 to 3.0 μM, which is comparable to the K_{d} of HA-IS of 0.6-1.1 μM. For the application of binding HA to remove PBUTs, the binding affinity between surface modification ligand and HA can be comparable to the affinity between HA and IS, leading to competition between surface ligand and IS for binding to HA.

Significantly different albumin binding behavior is seen at different stages of chemical functionalization of the SPR chip. Unmodified chips show a binding signal as high as 0.290 because of albumin fouling onto Au. Chips functionalized with PEGs (with terminus of —NH₂, —COOH, or —N₃) show signal levels around 0.05° due to the anti-fouling nature of PEG. Fully functionalized chips show absorption signals around 0.2° when fully occupied by HA. This lower shift (0.2°), compared to a bare Au signal (0.29°), is expected due to the increased distance of binding ligands from the Au plasmon surface when a PEG layer is present and limited density of binding sites.

FIG. 7 shows the reversible HA adsorption and desorption on functionalized SPR chips with 2 LA cycles and a final thiocyanide release cycle. SPR shift has a strong dependence on solution conditions, thus one can only compare shift signals after a buffer rinse. The initial sensor baseline is set to zero. For the first cycle, after initial HA exposure and buffer rinse a shift of 0.225 is seen corresponding to full HA coverage. After LA exposure and buffer rinse a drop in shift 0.122 is seen instead of the ideal zero if HA is completely released to initial state. The percent released is calculated as (0.225-0.122)/0.225 or ˜46%. The signal corresponding to protein adsorption was found to be similar over cycles which is an indication of stable binding affinity, which leads to similar amount of captured protein over cycles. Even though the total amount of captured protein remains stable, an increasing amount of un-released protein over cycles was observed. Un-releasable protein was expected due to fouling onto unprotected surface of desorbed surface chemistry and practical limitations of buffer rinse times. Thiol-gold is known to be unstable under oxidative conditions, therefore, functionalized SPR chips were found to have reduced release capacity after long storage time or testing with more than five adsorption/desorption cycles. SPR angle shifts and releasability percent as a function of surface functionalization and release chemical are summarized in Table 2.

Fatty acid-like ligands and ligands of longer hydrocarbon chain length show stronger binding affinity as octadecane (C18) shows a SPR signal 0.24°, which is comparable to a set reference of 0.25° representing full-surface coverage, while hexane (C6) shows a signal of 0.05°, which is near the detection limit. Octane and decane ligands show intermediate signals of 0.110 and 0.15°. Capric acid has same chain length compared to decane but shows higher binding signal of 0.180 due to its carboxyl terminus which further stabilizes the binding through electrostatic interaction. Longer alkane chain length can be desirable for better protein binding stability but undesirable for dissociation of protein from bound ligand. For instance, the octadecane surface shows significant binding to HA but the release agents proved to have limited effects. Balancing the binding and dissociation ability, capric acid can be thought to be the best performing fatty acid-like ligand.

Another set of ligands, including Cibacron Blue (CB), Direct Blue 71 (DB71), and tryptophan, that specifically target the S-II site of HA has large aromatic rings and charged side groups. The surface chemistry ligands were stabilized in the S-II cavity by hydrophobic-hydrophobic and electrostatic interactions and it-stacking between aromatic rings and surrounding moieties. The HA binding signal was found to be 0.25°, 0.19°, 0.170 and 0.19° for CB, DB71, tryptophan of —NH₂ and —COOH terminus respectively. Compared to fatty acid-targeting ligands, S-II site-targeting ligands showed a more stable binding of albumin. However, only CB showed feasible release ability for HA regeneration.

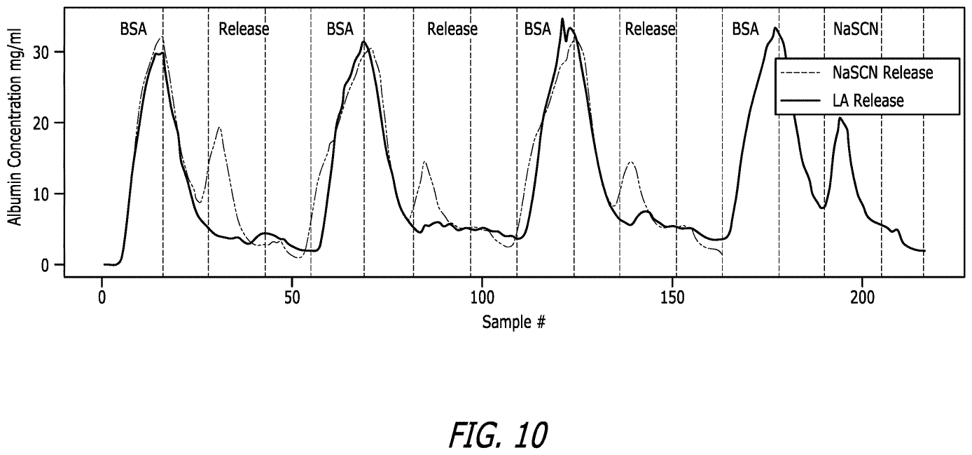

The interaction between albumin and Cibacron blue (CB) was initially investigated using a 2M NaSCN solution to assess the strength of bovine serum albumin (BSA) adsorption on the CB column, as depicted in FIG. 10. In contrast to another test using LA as the release agent (LA Release), the results of the adsorption-desorption test with NaSCN as the release agent (NaSCN Release) exhibited a distinct peak at the intended position. Numerical analysis of the test cycle curve unveiled the stability of albumin adsorption across all listed cycles, while the release agent NaSCN had a consistent ability to induce albumin desorption from the CB column over cycles. This pattern indicated that the albumin-Cibacron blue interaction is both stable and reversible.

Conversely, results from the LA release displayed a limited response to the release agent. Additionally, the albumin desorption peak from the last cycle using NaSCN was observed to be higher than the release peak from the red curve. This observation can be attributable to albumin accumulation from prior adsorption sessions. Without wishing to be bound by any particular theory, as the albumin-Cibacron blue interaction has demonstrated stability and reversibility, it can be thought that lower BSA release by LA can be attributed to the higher concentration of albumin employed, or the structural differences between BSA and human serum albumin (HAS), which was used in other experiments.

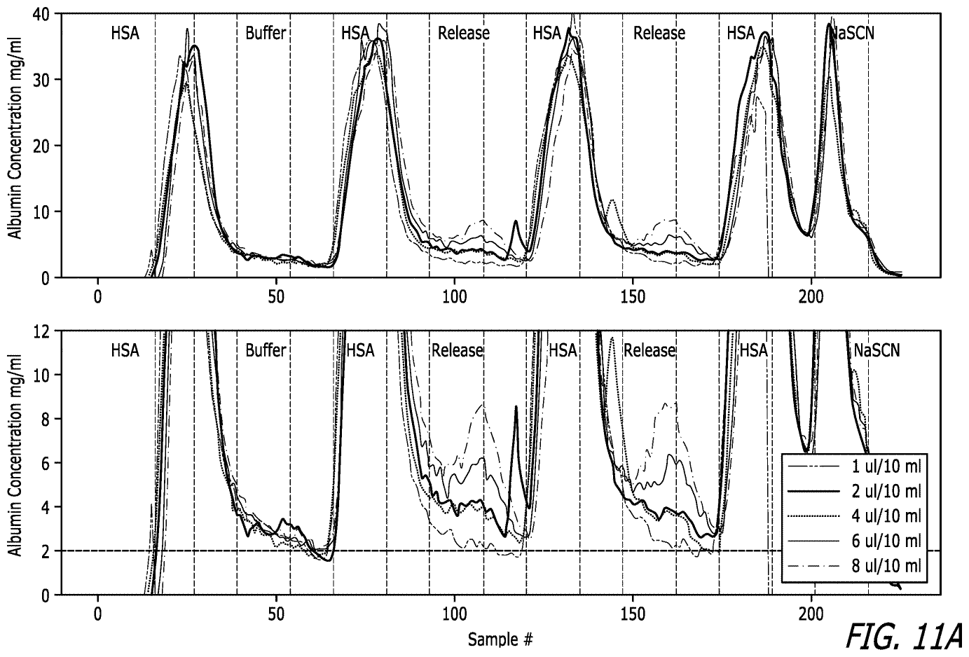

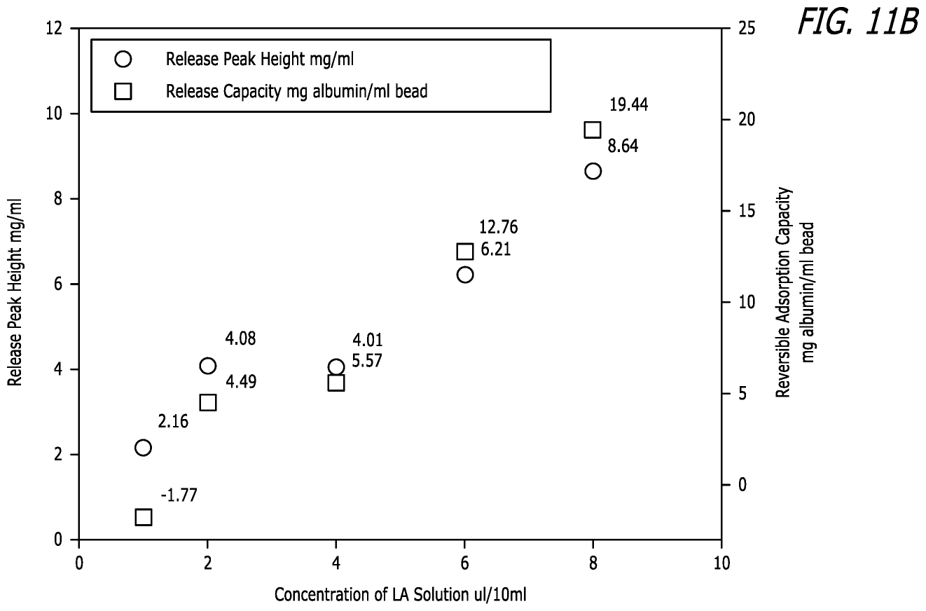

To test the aforementioned hypotheses, a series of experiments were conducted using LA solutions of varying concentrations and HSA solutions with a concentration of 40 mg/ml. As depicted at FIG. 11A, these experiments were carried out with a slight operational modification. The first adsorption-desorption cycle was executed with phosphate buffer serving as a mock release agent to reveal the actual desorption strength of the release agent.

Through a comparison between the release session with buffer and linoleic acid (LA) shown in FIG. 11A, it was evident that even a small addition of LA, as little as 2 μl/10 ml, induced the desorption of HSA from the CB column, although with a minor release effect. Increasing the LA solution concentration to 4 μl/10 ml did not exhibit significant improvements in release capability. However, when the LA solution concentration was further elevated to 6-8 μl/10 ml, a linear enhancement in HSA desorption performance was observed.

The amount of HSA released by LA was quantified by integrating the area beneath the release session curve and subtracting the baseline corresponding to the phosphate buffer rinse. This value represents the fraction of HSA that can be manipulated by the LA solution in a reversible manner. In this context, the adsorption of this fraction of HSA onto the Cibacron blue functionalized beads (CB beads) was considered reversible by LA. Subsequently, the reversible adsorption capacity of CB beads and the albumin release peak height, from FIG. 11A, were plotted against the LA solution concentration, as shown in FIG. 11B.

It is easy to deduce a linear relationship between LA solution concentration and the reversible adsorption capacity of CB beads or the release peak height after the LA solution concentration surpasses 4 μl/10 ml level. Notably, the threshold for significant HSA desorption is lower at 2 μl/10 ml, which is in alignment with the previous study. However, it was only through the increase of the LA solution concentration to 8 μl/10 ml that an HSA desorption capacity of 19.44 mg/ml beads was achieved, surpassing the value from the previous study, which stood at 15.3 mg/ml beads. The need for higher LA concentrations to achieve effective HSA release is likely a consequence of the elevated HSA concentration used in these tests. This is because the association between HSA and CB beads becomes stronger under these conditions.

Further improving the release capability of linoleic acid by increasing its concentration can be desirable. However, the LA “solution” was actually a micelle suspension acquired by ultrasonication. Such suspension of micelle is subject to stability challenges with increased concentration over long term. Furthermore, the previously used concentration of LA was physiologically equivalent. Increasing the use of LA can indeed enhance protein elution efficiency, but it also complicates the subsequent removal of LA. Therefore, considering that the demonstrated albumin desorption capability has already met the design requirements, and further elevating the LA concentration would complicate subsequent removal steps, further increasing the LA concentration was not evaluated.

Previous studies have shown that it is difficult to remove albumin bound to a CB chromatography column. Only NaSCN solutions with high concentrations near 2 M were found to sufficiently strip absorbed albumin from CB chromatography columns, which would be unacceptable for direct contact with blood plasma. As is shown in Table 2, a variety of chemicals were chosen to release albumin by different mechanisms. NaSCN works as a chaotropic agent disrupting H-bonds, protein-ligand interactions, and protein conformation. NaCl and Na₂SO₄ of high ionic strength shields the electrostatic interaction between ligand and albumin. The pH 3.6 buffer protonates the albumin therefore altering its charge in order to release it from the surface. Fatty acids and SDS target fatty acid binding sites to competitively replace surface ligands attached to albumin to initiate release. Ibuprofen (ibp) competes with surface ligands to occupy the S-II site. causing release. Despite showing the best albumin release ability, SCN— is not a viable choice for HA regeneration because of its toxicity and that large volumes of ion exchange solution are needed downstream. SDS effectively releases albumin but also denatures it. In order to demonstrate the feasibility of a portable albumin regeneration device, the release chemical used must be biocompatible or easy to remove in downstream processes. High ionic strength and pH-altering buffer solutions were considered for ease of downstream reversal. However, both failed to provide satisfying release capacity, as shown in Table 2. Biocompatible fatty acids were then examined. Plasma-prevalent linoleic acid (LA) and the LA-containing fatty acid mixture showed great albumin release capacity of 44.1% and 49.5% on CB surface even at limited concentrations of about 0.5 μg/mL.

In Table 3, the optimal albumin binding chemistry and release conditions were measured. The release capacities were calculated with respect to their monolayer averaged SPR signal shift (Cibacron Blue: 0.23°, n=76; Capric Acid: 0.14°, n=50). From fatty acid binding ligands, capric acid was found to have the best adsorption/desorption capacity. CB is the best for albumin regeneration among the S-II targeting binding ligands. On both types of treated sensor surface, linoleic acid and fatty acid mix was able to release approximately ⅓ of a monolayer-worth of bound protein, which allows the cleaned albumin and the release agent to be returned to plasma without further treatment.

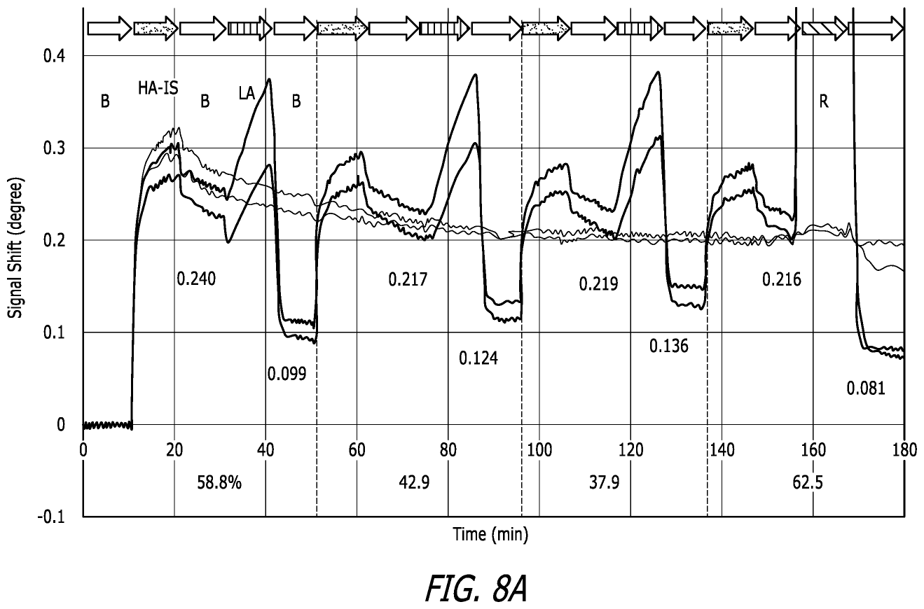

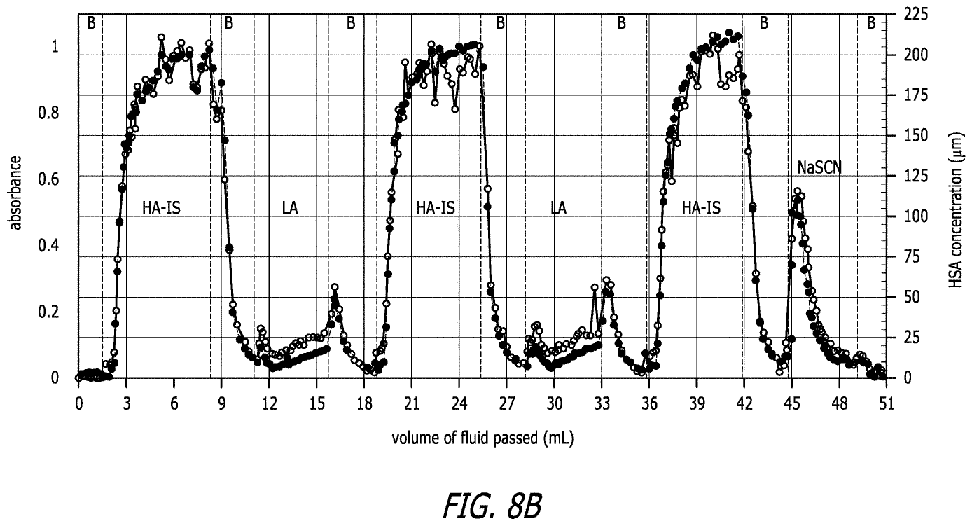

There is a risk that HA carrying PBUT in a S-II site will not bind to the functionalized surfaces. Binding HA-PBUT complex to a surface can result in release of the PBUT. As shown in FIG. 8A, the HA-IS binding-induced SPR signal shifts are comparable to those of the tests that used albumin with unoccupied S-II sites. This indicates the HA binding affinity to CB-grafted surface does not change with pre-occupied S-IL site. The captured HA-IS complex can be desorbed with the same fatty acid release chemistry.

To demonstrate practical application for clinically relevant amounts of HA-PBUT cleansing, commercially available CB-functionalized agarose beads were purchased and built into an adsorption column as described in the Methods section. In cycles, phosphate buffer, HA-IS complex solution, phosphate buffer, and then release agents (linoleic acid or NaSCN) were pumped through the column and the eluent was collected. CB-functionalized beads were expected to adsorb HA-IS and cause the dissociation of IS from the captured HA. Release agents were expected to cause desorption of captured IS-free HA from the adsorption column. These adsorption and desorption behaviors were studied by optical absorbance at 280 nm of the eluent proportional to HA-IS concentration. As FIG. 8B shows, with low initial eluent concentration rising to the feed concentration, HA-IS complex quickly binds to and saturates CB-agarose beads. Then both LA and NaSCN release HA from the adsorption column. However, the release behavior was found to be different between LA and NaSCN. On one hand, NaSCN shows fast release immediately upon introduction which corresponds to a sharp peak in FIG. 8B. On the other hand, LA shows most release occurs at the buffer rinse after LA flow. Without wishing to be bound by theory, it is thought that the delayed LA release was caused by LA micelles dissolving at the buffer solution exchange front where concentration drops below the critical micelle concentration (0.1 mM) giving more free-floating molecules of LA capable of release. This release profile may be optimized by multiple shorter buffer/LA cycles. Even though it is clear from the absorption measurements that HA or HA-IS is bound by the CB chemistry and then eventually released when a release agent is flowed through, whether IS was removed from HA upon binding was not clear, as the absorbance includes contributions from both IS and HA, and so they cannot be distinguished from this data alone.

Bicinchoninic acid (BCA) assay is a protein sensitive quantification method which was used in this example to measure the concentration of HA. From the BCA assay curves it was possible to calculate binding capacity of the column, which was 0.371, 0.304, 0.371 mol HA/mL beads corresponding to three cycles of adsorption/desorption, shown in FIG. 8B. Similarly, release capacities were measured to be: 0.177 (LA), 0.232 (LA), 0.292 (NaSCN) μmol HA/mL beads for the three cycles. Specifications for the CB-functionalized beads have 0.271 mol/mL beads, so the experimental release capacities are close to expected values. Binding capacity can be easily over-estimated because it is hard to precisely determine the dead volume and starting time of flow across an adsorption column. From these calculations, LA releases at least 60% (or up to 79%) as well as commercially used NaSCN. Comparing binding capacity to release capacity. LA and NaSCN release at least 47% and 79% of HA bound to the column, respectively. Both values corroborate SPR results (31% and 65%, respectively). LA shows a promising release capacity, though lower than NaSCN, for a CB-based HA regeneration system because, unlike NaSCN, LA is plasma-compatible.

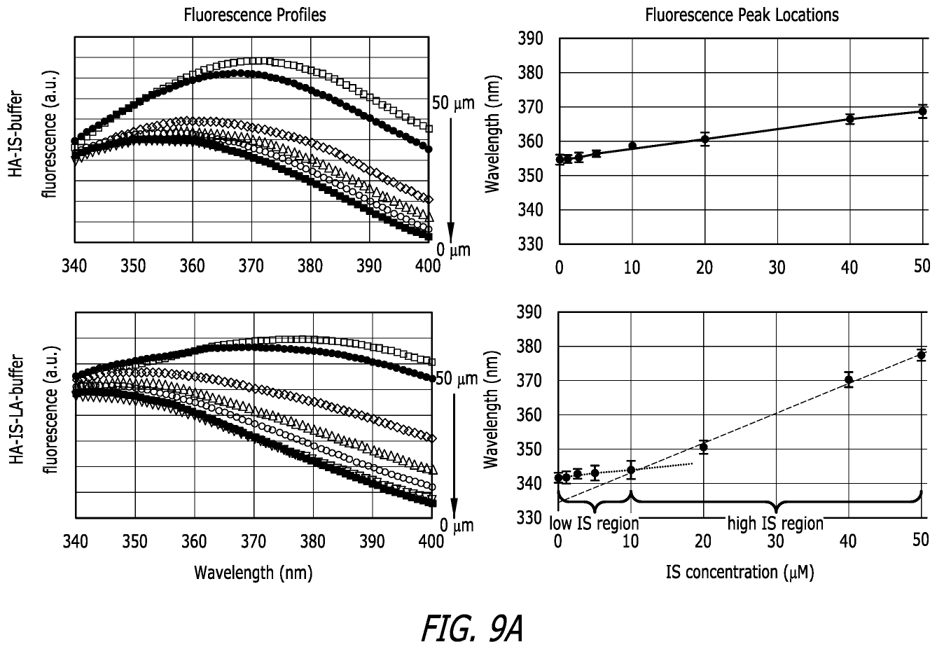

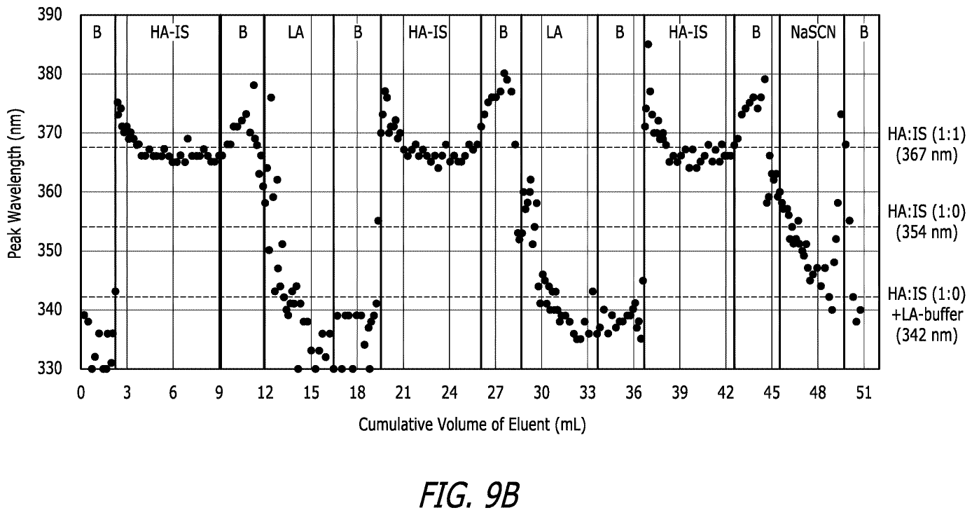

Even though it is demonstrated that HA capture and release was achieved with the plasma-compatible CB-LA chemistry, the absorption and BCA assay studies do not prove that binding of HA causes IS release, which is important for toxin separations in treatment. Theoretically, it is possible to calculate IS concentration from subtraction of BCA-derived HA concentration from total solution absorbance and using known optical absorption coefficients. However, the system proved too noisy to produce conclusive results in an analysis that subtracts between two measurements. Addressing this problem, fluorescence measurements were used to distinguish HA from IS. HA fluorescence is based on the tryptophan residue in S-II binding site which can be influenced by its surrounding chemical environment, such as the bound IS, release agent and composition of buffer solution. Spectra measurements were taken of HA-IS solutions with and without LA (FIG. 9A).

In the case of simple HA-IS mixture solutions, going from 1:0 to 1:1 ratio, there is a linear peak shift of a single fluorescence peak wavelength from 354 to 367 nm consistent with a coupled IS in S-II site. While in the presence of LA, the HA-IS fluorescence spectrum includes two independent peaks with the HA peak shifted down to 342 nm and IS peak appearing at 380 nm as IS concentration increases. This behavior is consistent with the hypothesis that HA forms an HA-LA complex at the S-II site, causing a shift in HA fluorescence peak wavelength and formation of an independent IS peak. Importantly, it is shown that released HA is cleaned and contains no IS (<10 μM detection limit) with an observed fluorescence peak at 342 nm.

FIG. 9B shows the fluorescence peak wavelength as a function of volume of solution eluted through the column. At initial HA-IS introduction, there is a temporary shift to −380 nm consistent with free IS released as HA is bound to beads. This behavior indicates that the IS was removed from HA when the CB-chemistry captured the HA-IS complex. The peak then shifts to the 1:1 wavelength of 367 nm for the feed solution flowing over saturated beads. The second rinse also causes a jump to ˜380 nm, presumably rinsing off loosely bound IS on other HA sites. Notably, when LA is added the peaks shifted to 342 nm, showing that IS-free HA was released. Thus, IS was removed from HA and HA was regenerated.

BCA protein analysis was used to quantify HA concentration while fluorescence peak shift analysis showed that the released HA was cleaned of IS. These analyses showed that LA has ˜60% of the release capacity of the NaSCN standard. Release capacity was measured to be ˜0.2 μmol HA/mL beads (13.5 mg/mL). In the disclosed example system, flow rate was set to 4.0 mL solution/min. Based on the data, a column is saturated with bound HA in ˜1 min. and full release could take as little as 1 min. if LA and buffer cycles were optimized to maximize release. Given 2 min. binding/release cycles, 120 cycles would be possible in a 4-hour conventional dialysis treatment session. To calculate the volume of chromatography resin required for such processing one can use the simple relationship Resin volume=A/PC where A is the amount of HA to process per session (250 g), P is the processing capacity per cycle (13.5 mg/mL), and C is the number of cycles (120). To process the typical adult amount of HA in blood plasma (250 g) in one 4-hour hemodialysis session, only 155 mL chromatography resin is needed, enabling a practical dialysis treatment design.

### CONCLUSIONS

Presently there are no treatment options to reduce protein bound toxins to non-uremic levels. A possible approach is to bind HA-PBUT complexes to a surface, release toxin to a waste stream/dialyzer, and then release HA back into the plasma stream. Currently used HA purification methods use toxic levels of NaSCN requiring extensive ion exchange volumes not compatible with dialysis treatments. Herein it is disclosed that that plasma compatible fatty acids (e.g., linoleic acid and fatty acid mixture at physiological ratios) can release cleaned HA and be returned to a plasma stream for potential dialysis treatment. SPR studies showed Cibacron Blue and capric acid to be suitable surface functionalization for HA capture and release, while long chain alkanes bind too strongly resulting in low release yield. The process was scaled to commercially available Cibacron Blue-functionalized agarose beads commonly used for HA purification by chromatography. Binding capacities of 0.35 gmol HA/mL beads and release amounts of 0.23 μmol HA/mL beads were observed using the plasma compatible linoleic acid. Fluorescence studies of the released HA eluents showed the HA-IS complex had been stripped of the protein bound toxin indoxyl sulfate. During a conventional 4-hour dialysis session with 2-minute binding release cycles, only 155 mL of CB-agarose beads would be needed to process the 250 g of HA in a typical patient. This new process can enable more effective dialysis treatments and positively impact mortality rates associated with heart disease in hemodialysis patients.

### NON-LIMITING EMBODIMENTS

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.

Embodiment 1. A dialysis system, comprising: an immobilized ligand configured to competitively bind a toxin binding site of a protein and displace a toxin from the toxin binding site of the protein into a first eluate, wherein the dialysis system is configured for return of the protein to blood of a subject.

Embodiment 2. The dialysis system of any other Embodiment, further comprising a plasma fraction membrane cartridge, positioned upstream of the protein-bind column, configured to divert a portion of plasma of whole blood of the subject into the plasma fluid stream.

Embodiment 3. The dialysis system of any other Embodiment, further comprising: a biocompatible release agent configured to release the protein from the immobilized ligand to a second eluate for return of the protein to blood of a subject; and a biocompatible release agent filter configured to decrease a concentration of the biocompatible release agent in the second eluate.

Embodiment 4. The dialysis system of any other Embodiment, wherein the uremic toxin comprises an aromatic-anion structure, p-cresol sulfate, hippuric acid, bilirubin, an indole derivative, indoxyl sulfate (IS), p-cresyl sulfate, any derivative thereof, or any combination thereof.

Embodiment 5. The dialysis system of any other Embodiment, wherein the immobilized ligand comprises Cibacron Blue (CB), Direct Blue 71, tryptophan with N-terminus functionalized, tryptophan with C-terminus functionalized, octadecane, decane, capric acid, octane, hexane, any derivative thereof, or any combination thereof.

Embodiment 6. The dialysis system of any other Embodiment, wherein the biocompatible release agent comprises a fatty acid, linoleic acid (LA), a fatty acid mix, any derivative thereof, or any combination thereof.

Embodiment 7. The dialysis system of any other Embodiment, wherein the biocompatible release agent is an inhomogeneous emulsion comprising LA, oleic acid, palmitic acid, any derivative thereof, or any combination thereof.

Embodiment 8. The dialysis system of any other Embodiment, further comprising a dialyzer configured for small molecule hemodialysis of the first eluate, the second eluate, or both, into a regenerated dialysate loop.

Embodiment 9. The dialysis system of any other Embodiment, further comprising a small molecular weight (SMW) cutoff filter configured for recirculation of bloodborne small molecular weight compounds through the dialysis system.

Embodiment 10. The dialysis system of any other Embodiment, wherein the dialyzer is configured for combined dialysis of a stream that includes post-toxin removal plasma and whole blood.

Embodiment 11. The dialysis system of any other Embodiment, wherein the dialyzer is configured for separated dialysis of a first stream that includes post-toxin removal plasma and a second stream that includes whole blood, wherein the first stream and the second stream are combined after flow through the dialyzer.

Embodiment 12. The dialysis system of any other Embodiment, wherein the dialysis system is an after-market add-on that is compatible with a system that comprises the dialyzer.

Embodiment 13. The dialysis system of any other Embodiment, wherein the dialyzer is integral with the dialysis system as a unit.

Embodiment 14. A dialysis system, comprising: a protein-bind column operably connectable to a plasma fluid stream at an inlet thereof and at an outlet thereof, wherein the protein-bind column comprises a resin that comprises an immobilized ligand configured to bind a toxin binding site of an albumin protein and displace a uremic toxin from the toxin binding site of the albumin protein into a first eluate; and a plasma fraction membrane cartridge, positioned upstream of the protein-bind column, configured to divert a portion of plasma of whole blood of a subject into the plasma fluid stream, wherein the dialysis system is configured for return of the albumin protein to blood of the subject.

Embodiment 15. The dialysis system of any other Embodiment, further comprising: a biocompatible release agent configured to release the albumin protein from the immobilized ligand to a second eluate; and a biocompatible release agent filter configured to decrease a concentration of the biocompatible release agent in the second eluate.

Embodiment 16. The dialysis system of any other Embodiment, wherein the uremic toxin comprises an aromatic-anion structure, p-cresol sulfate, hippuric acid, bilirubin, an indole derivative, indoxyl sulfate (IS), p-cresyl sulfate, any derivative thereof, or any combination thereof.

Embodiment 17. The dialysis system of any other Embodiment, wherein the immobilized ligand comprises Cibacron Blue (CB). Direct Blue 71, tryptophan with N-terminus functionalized, tryptophan with C-terminus functionalized, octadecane, decane, capric acid, octane, hexane, any derivative thereof, or any combination thereof.

Embodiment 18. The dialysis system of any other Embodiment, wherein the biocompatible release agent comprises a fatty acid, linoleic acid (LA), a fatty acid mix, any derivative thereof, or any combination thereof.

Embodiment 19. The dialysis system of any other Embodiment, wherein the biocompatible release agent is an inhomogeneous emulsion comprising LA, oleic acid, palmitic acid, any derivative thereof, or any combination thereof.

Embodiment 20. The dialysis system of any other Embodiment, further comprising a dialyzer configured for small molecule hemodialysis of the first eluate, the second eluate, or both, into a regenerated dialysate loop.

Embodiment 21. The dialysis system of any other Embodiment, further comprising a small molecular weight (SMW) cutoff filter configured for recirculation of bloodborne small molecular weight compounds through the dialysis system.

Embodiment 22. The dialysis system of any other Embodiment, wherein the dialyzer is configured for combined dialysis of a stream that includes post-toxin removal plasma and whole blood.

Embodiment 23. The dialysis system of any other Embodiment, wherein the dialyzer is configured for separated dialysis of a first stream that includes post-toxin removal plasma and a second stream that includes whole blood, wherein the first stream and the second stream are combined after flow through the dialyzer.

Embodiment 24. The dialysis system of any other Embodiment, wherein the dialysis system is an after-market add-on that is compatible with a system that comprises the dialyzer.

Embodiment 25. The dialysis system of any other Embodiment, wherein the dialyzer is integral with the dialysis system as a unit.

Embodiment 26. A hemodialysis method, comprising: separating a toxic plasma fraction from whole blood of a subject in need of dialysis; binding a toxin-protein complex of the toxic plasma fraction to an immobilized ligand; separating a toxin from the toxin-protein complex such that the toxin flows to a first eluate and a protein of the toxin-protein complex remains bound to the immobilized ligand: dialyzing the first eluate to remove the toxin and produce a clean first eluate; eluting the protein from the immobilized ligand with a biocompatible release agent such that the protein flows to a second eluate; removing at least a portion of the biocompatible release agent from the second eluate to form a clean second eluate; dialyzing and combining the clean first eluate and the clean second eluate to produce a clean plasma fraction; and returning at least a portion of the clean plasma fraction to blood of the subject.

Embodiment 27. The hemodialysis method of any other Embodiment, wherein the uremic toxin comprises an aromatic-anion structure, p-cresol sulfate, hippuric acid, bilirubin, an indole derivative, indoxyl sulfate (IS), p-cresyl sulfate, any derivative thereof, or any combination thereof.

Embodiment 28. The hemodialysis method of any other Embodiment, wherein the immobilized ligand comprises Cibacron Blue (CB). Direct Blue 71, tryptophan with N-terminus functionalized, tryptophan with C-terminus functionalized, octadecane, decane, capric acid, octane, hexane, any derivative thereof, or any combination thereof.

Embodiment 29. The hemodialysis method of any other Embodiment, wherein the biocompatible release agent comprises a fatty acid, linoleic acid (LA), a fatty acid mix, any derivative thereof, or any combination thereof.

Embodiment 30. The hemodialysis method of any other Embodiment, wherein the biocompatible release agent is an inhomogeneous emulsion comprising LA, oleic acid, palmitic acid, any derivative thereof, or any combination thereof.

Embodiment 31. A composition comprising the clean plasma fraction of any other Embodiment.

Embodiment 32. A method for treating end stage renal disease (ESRD), the method comprising administering the composition of Embodiment 31 to a subject in need thereof.

While illustrative embodiments have been illustrated and described, it will be appreciated that various changes can be made therein without departing from the spirit and scope of the disclosure.

1. A dialysis system, comprising:
an immobilized ligand configured to competitively bind a toxin binding site of a protein and displace a toxin from the toxin binding site of the protein into a first eluate, wherein the dialysis system is configured for return of the protein to blood of a subject.

2. The dialysis system of claim 1, further comprising a plasma fraction membrane cartridge, positioned upstream of the protein-bind column, configured to divert a portion of plasma of whole blood of the subject into the plasma fluid stream.

3. The dialysis system of claim 1, further comprising:
a biocompatible release agent configured to release the protein from the immobilized ligand to a second eluate for return of the protein to blood of a subject; and
a biocompatible release agent filter configured to decrease a concentration of the biocompatible release agent in the second eluate.

4. The dialysis system of claim 1, wherein the uremic toxin comprises an aromatic-anion structure, p-cresol sulfate, hippuric acid, bilirubin, an indole derivative, indoxyl sulfate (IS), p-cresyl sulfate, any derivative thereof, or any combination thereof.

5. The dialysis system of claim 1, wherein the immobilized ligand comprises Cibacron Blue (CB), Direct Blue 71, tryptophan with N-terminus functionalized, tryptophan with C-terminus functionalized, octadecane, decane, capric acid, octane, hexane, any derivative thereof, or any combination thereof.

6. The dialysis system of claim 1, wherein the biocompatible release agent comprises a fatty acid, linoleic acid (LA), a fatty acid mix, any derivative thereof, or any combination thereof.

7. (canceled)

8. The dialysis system of claim 1, further comprising a dialyzer configured for small molecule hemodialysis of the first eluate, the second eluate, or both, into a regenerated dialysate loop.

9. The dialysis system of claim 8, further comprising a small molecular weight (SMW) cutoff filter configured for recirculation of bloodborne small molecular weight compounds through the dialysis system.

10-13. (canceled)

14. A dialysis system, comprising:
a protein-bind column operably connectable to a plasma fluid stream at an inlet thereof and at an outlet thereof, wherein the protein-bind column comprises a resin that comprises an immobilized ligand configured to bind a toxin binding site of an albumin protein and displace a uremic toxin from the toxin binding site of the albumin protein into a first eluate; and
a plasma fraction membrane cartridge, positioned upstream of the protein-bind column, configured to divert a portion of plasma of whole blood of a subject into the plasma fluid stream, wherein the dialysis system is configured for return of the albumin protein to blood of the subject.

15. The dialysis systems of claim 14, further comprising:
a biocompatible release agent configured to release the albumin protein from the immobilized ligand to a second eluate; and
a biocompatible release agent filter configured to decrease a concentration of the biocompatible release agent in the second eluate.

16. The dialysis system of claim 14, wherein the uremic toxin comprises an aromatic-anion structure, p-cresol sulfate, hippuric acid, bilirubin, an indole derivative, indoxyl sulfate (IS), p-cresyl sulfate, any derivative thereof, or any combination thereof.

17. The dialysis system of claim 14, wherein the immobilized ligand comprises Cibacron Blue (CB), Direct Blue 71, tryptophan with N-terminus functionalized, tryptophan with C-terminus functionalized, octadecane, decane, capric acid, octane, hexane, any derivative thereof, or any combination thereof.

18. The dialysis system of claim 14, wherein the biocompatible release agent comprises a fatty acid, linoleic acid (LA), a fatty acid mix, any derivative thereof, or any combination thereof.

19. (canceled)

20. The dialysis system of claim 14, further comprising a dialyzer configured for small molecule hemodialysis of the first eluate, the second eluate, or both, into a regenerated dialysate loop; and
a small molecular weight (SMW) cutoff filter configured for recirculation of bloodborne small molecular weight compounds through the dialysis system.

21-25. (canceled)

26. A hemodialysis method, comprising:
separating a toxic plasma fraction from whole blood of a subject in need of dialysis;
binding a toxin-protein complex of the toxic plasma fraction to an immobilized ligand;
separating a toxin from the toxin-protein complex such that the toxin flows to a first eluate and a protein of the toxin-protein complex remains bound to the immobilized ligand;
dialyzing the first eluate to remove the toxin and produce a clean first eluate;
eluting the protein from the immobilized ligand with a biocompatible release agent such that the protein flows to a second eluate;
removing at least a portion of the biocompatible release agent from the second eluate to form a clean second eluate;
dialyzing and combining the clean first eluate and the clean second eluate to produce a clean plasma fraction; and
returning at least a portion of the clean plasma fraction to blood of the subject.

27. The hemodialysis method of claim 26, wherein the uremic toxin comprises an aromatic-anion structure, p-cresol sulfate, hippuric acid, bilirubin, an indole derivative, indoxyl sulfate (IS), p-cresyl sulfate, any derivative thereof, or any combination thereof.

28. The hemodialysis method of claim 26, wherein the immobilized ligand comprises Cibacron Blue (CB), Direct Blue 71, tryptophan with N-terminus functionalized, tryptophan with C-terminus functionalized, octadecane, decane, capric acid, octane, hexane, any derivative thereof, or any combination thereof.

29. The hemodialysis method of claim 26, wherein the biocompatible release agent comprises a fatty acid, linoleic acid (LA), a fatty acid mix, any derivative thereof, or any combination thereof.

30. (canceled)

31. A composition comprising the clean plasma fraction of claim 26.

32. A method for treating end stage renal disease (ESRD), the method comprising administering the composition of claim 31 to a subject in need thereof.
